(19) United States
(12) Patent Application Publication
(10) Pub. No.: US 2011/0275701 A1
(43) Pub. Date: Nov. 10, 2011

(54) ANTI-THROMBIN APTAMER FORMULATIONS AND METHODS FOR USE

(75) Inventor: Renta Hutabarat, Winchester, MA (US)

(73) Assignee: Archemix Corp.

(21) Appl. No.: 13/119,826

(22) PCT Filed: Sep. 11, 2009
(86) PCT No.: PCT/US09/05117

§ 371 Date: Jun. 3, 2011

Related U.S. Application Data
(60) Provisional application No. 61/192,578, filed on Sep. 18, 2008.
(60) Provisional application No. 61/192,629, filed on Sep. 18, 2008.

(51) Int. Cl. A61K 31/7088; A61P 7/02; A61P 13/12; C07H 21/04

(52) U.S. Cl. 514/44 R; 536/23.2

(57) ABSTRACT

The invention relates to the formulation, dosing, administration and use of an aptamer antagonist therapeutic that binds to thrombin.

### CROSS-REFERENCE TO RELATED APPLICATIONS

This application claims the benefit under 35 USC §119(e)(1) of U.S. Provisional Application Nos. 61/192,578, filed Sep. 18, 2008 and 61/192,629, filed Sep. 18, 2008, which applications are incorporated herein by reference in their entireties.

### FIELD OF THE INVENTION

The invention relates to the formulation, dosing, administration and use of an aptamer antagonist therapeutic that binds to thrombin.

### BACKGROUND OF THE INVENTION

### Thrombin

Thrombin is a multi-functional serine protease that has procoagulant and anticoagulant activities. As a procoagulant enzyme, thrombin activates fibrinogen, platelets and clotting factors V, VIII, and XIII. The specific cleavage of fibrinogen by thrombin initiates the polymerization of fibrin monomers, a primary event in blood clot formation. The central event in the formation of platelet thrombi is the activation of platelets from the “nonbinding” mode to the “binding” mode. Thrombin is a physiologic activator of platelet aggregation. Thus, as a procoagulant, thrombin plays a key role in the arrest of bleeding (physiologic hemostasis) and formation of vaso-occlusive thrombi (pathologic thrombosis).

As an anticoagulant, thrombin binds to thrombomodulin, a glycoprotein expressed on the surface of vascular endothelial cells. Thrombomodulin alters substrate specificity from fibrinogen and platelets to protein C through a combination of an allosteric change in the active site conformation and an overlap of the thrombomodulin and fibrinogen binding sites on thrombin. Activated protein C, in the presence of a phospholipid surface, Ca²⁺, and a second vitamin K-dependent protein cofactor, protein S, inhibits coagulation by proteolytically degrading clotting factors Va and VIIIa. Thus, the formation of the thrombin-thrombomodulin complex converts thrombin from a procoagulant to an anticoagulant enzyme, and the normal balance between these opposing activities is critical to the regulation of hemostasis.

### Coagulation Disorders

Vascular injury and thrombus formation represent the key events in the pathogenesis of various vascular diseases, including atherosclerosis. The pathogenic processes in the activation of platelets and/or the clotting system, leading to thrombosis in various disease states and in various sites, such as the coronary arteries, cardiac chambers and prosthetic heart valves, appear to be different. Therefore, the use of a platelet inhibitor, an anticoagulant or a combination of both may be required in conjunction with thrombolytics to open vessels that are closed and prevent reocclusion.

Controlled proteolysis by compounds of the coagulation cascade is critical for hemostasis. As a result, a variety of complex regulatory systems exist that are based, in part, on a series of highly specific protease inhibitors. In a pathological situation, functional inhibitory activity can be interrupted by excessive production of active protease or inactivation of inhibitory activity. Perpetuation of inflammation in response to trauma (tissue damage) or infection (sepsis) depends on proteolytic enzymes, both of plasma cascade systems, including thrombin, and of lysosomal origin. Multiple organ failure in these cases is enhanced by the concurrently arising imbalance between proteases and their inhibitory regulators. In addition, an imbalance of thrombin activity in the brain may lead to neurodegenerative diseases.

### Coronary Artery Bypass Graft (CABG) Surgery

In 2001, the American Heart Association reported that an estimated 12.4 million people in the United States were diagnosed with some form of coronary artery disease. Given thrombin's importance in the coagulation process, an anti-thrombin agent or thrombin inhibitor is the anticoagulant used, e.g., during coronary artery bypass graft (CABG) surgery, percutaneous coronary intervention (hereinafter “PCI”) and acute coronary syndrome. As of 2001, more than 570,000 CABG procedures were performed annually in the U.S. and it is estimated that over 700,000 procedures are performed worldwide. Currently, the most commonly used anticoagulant is heparin, which must be used with the antidote protoamine. However, heparin-protoamine treatment is associated with a number of serious side-effects including bleeding and thrombocytopenia (platelet count reduction), which is often asymptomatic but may be associated with life-threatening arterial or venous thrombosis. In addition, heparin-protoamine treatment has a number of other disadvantages including: non-specific binding to plasma proteins, which results in resistance in some patients; heparin cannot inhibit clot-bound thrombin; heparin has non-linear kinetics making dosing difficult to control; and heparin is manufactured from beef or pork tissues, which have an inherent safety risk arising from the possibility for transmission of viruses and/or prions. Consequently, a number of newer, higher-cost anticoagulants, such as low molecular weight heparins and ANGIOMAX®, have gained significant penetration into this market. However, these compounds have similar side-effects and their anticoagulation activity cannot be rapidly reversed.

### Cardio-Pulmonary Bypass Surgery

Unfractionated heparin is the standard anticoagulant used during cardio-pulmonary bypass. Unfractionated heparin functions primarily and indirectly by activating anti-thrombin III. Advantages of unfractionated heparin compared to other anticoagulants include rapid onset, point of care monitoring and rapid reversal with protamine. However, heparin can promote platelet activation and dysfunction as well as heparin-induced thrombocytopenia and paradoxical thrombosis. The relatively short half-life of protamine (approximately 4.5 minutes) can result in heparin rebound and post-operative bleeding.

Thus, there is a significant unmet medical need for a safe, moderate-cost anticoagulant that does not require a separate reversing agent that is not associated with the side effects and disadvantages listed above. Accordingly, it would be beneficial to develop inhibitors of thrombin for use as therapeutics in the treatment of coagulation-related disorders that produce rapid and predictable onset and offset of anticoagulation without the need for an antidote.

### SUMMARY OF THE INVENTION

The present invention provides pharmaceutical compositions or formulations of aptamers that bind to thrombin, referred to herein as “anti-thrombin aptamers”, and methods for using such anti-thrombin aptamers to treat thrombin-mediated diseases and disorders, including the treatment of coagulation related disorders involving thrombin-mediated platelet coagulation, for decreasing or inhibiting thrombin-mediated coagulation and causing anticoagulation in a subject. The formulations comprise an anti-thrombin aptamer or a pharmaceutically acceptable salt thereof, and a pharmaceutically acceptable solvent. The formulations and dosages described herein are designed to maximize clinical efficacy in the treatment of thrombin-mediated diseases and disorders while simultaneously decreasing or minimizing adverse side effects, without the need for an antidote.

The formulations described herein comprise an anti-thrombin aptamer or a pharmaceutically acceptable salt thereof. The formulations may comprise any aptamer that binds to thrombin or a variant or a fragment thereof. Preferably, the aptamer binds to exosite 1 of thrombin.

Preferably, the anti-thrombin aptamer binds to thrombin or a fragment thereof and acts as an antagonist to inhibit the function of thrombin.

Preferably, the anti-thrombin aptamer is ARC2172, which is also known as NU172. ARC2172 is a synthetically manufactured DNA aptamer.

ARC2172 is an aptamer having the following structure:

| | |
|---|---|
| CGCCTAGGTTGGGTAGGGTGGTGGCG. | (SEQ ID NO: 1) |

The formulations may comprise any amount of anti-thrombin aptamer. Typically, the formulations comprise 10-100 mg/ml of anti-thrombin aptamer or a pharmaceutically acceptable salt thereof. For example, suitable concentrations of anti-thrombin aptamer or a pharmaceutically acceptable salt thereof include, but are not limited to, 10-100 mg/ml or any 0.1 mg/ml increment thereof. Preferably, formulations comprise 10-50 mg/ml of anti-thrombin aptamer or a pharmaceutically acceptable salt thereof. More preferably, formulations comprise 10-30 mg/ml of anti-thrombin aptamer or a pharmaceutically acceptable salt thereof. Even more preferably, formulations comprise 15-30 mg/ml of anti-thrombin aptamer or a pharmaceutically acceptable salt thereof. Most preferably, formulations comprise 15-25 mg/ml of anti-thrombin aptamer or a pharmaceutically acceptable salt thereof.

The formulations also comprise a pharmaceutically acceptable solvent. Preferably, the pharmaceutically acceptable solvent is selected from the group consisting of 0.9% saline (also known as physiological saline or sterile isotonic saline solution) or phosphate buffered saline. Most preferably, the pharmaceutically acceptable solvent is 0.9% saline.

The formulations may comprise any amount of pharmaceutically acceptable solvent.

Various embodiments of the formulations may, optionally, include one or more of the following: buffer, pH adjuster, tonicity agent, cosolvent or pharmaceutically acceptable carrier.

The total dose of anti-thrombin aptamer is administered so as to achieve and then maintain a steady state blood concentration equal to at least the EC₉₀, which is the concentration of drug that leads to 90% maximal response, and preferably of 10-35 μg/ml. The steady state blood concentration can be any concentration between, and including, 10 μg/ml and 35 μg/ml in increments of 0.1 μg/ml. Preferably, the total dose achieves a steady state blood concentration of 15-25 μg/ml. More preferably, the total dose achieves a steady state blood concentration of 18-22 μg/ml. Most preferably, the total dose achieves a steady state blood concentration of 20 μg/ml.

The total dose of anti-thrombin aptamer is administered in mg per kg (mg/kg) of body weight. The total dose of anti-thrombin aptamer is 1-30 mg/kg. The total dose can be any dose between, and including, 1.0 mg/kg and 30.0 mg/kg in increments of 0.1 mg/kg. This total dosage may be administered in a single dose, multiple doses, as a continual dose or a combination thereof.

Preferably, a total dose of anti-thrombin aptamer of 7-25 mg/kg is administered. More preferably, a total dose of 9-20 mg/kg is administered. Even more preferably, a total dose of 11-16 mg/kg is administered. Most preferably, a total dose of 12-15 mg/kg is administered. This total dosage may be administered in a single dose, multiple doses, as a continual dose or a combination thereof. For example, a loading dose or doses may be administered followed by a maintenance dose or doses. To achieve the desired blood concentration level, any total dose of 1.0-30.0 mg/kg can be used.

Preferably, the formulations are administered with a loading dose and a maintenance dose.

Preferably, the loading dose is a bolus intravenous infusion of anti-thrombin aptamer. For example, the loading dose is 0.2-2.5 mg/kg. More preferably, the loading dose is 0.75-2.25 mg/kg. Even more preferably, the loading dose is 1.0-2.0 mg/kg. Most preferably, the loading dose is 1.25-1.75 mg/kg. However, the dose and duration may be varied to achieve essentially the same result of a safe and tolerable dose that rapidly achieves the desired steady state concentration.

Preferably, the maintenance dose of anti-thrombin aptamer is administered as a continual infusion at a constant rate for four hours. Preferably, the maintenance dose is 6.0-25.0 mg/kg. More preferably, the maintenance dose is 8.0-18.0 mg/kg. Even more preferably, the maintenance dose is 10.0-16.0 mg/kg. Most preferably, the maintenance dose is 12.0-14.0 mg/kg. Preferably, the maintenance dose is administered as a continuous infusion until anticoagulation is no longer needed.

ARC2172 is manufactured for clinical use as a sterile isotonic saline solution (0.9% saline solution) for injection. Preferably, the formulation is provided in a 15 mg/mL solution. Preferably, the formulation is at pH 7.0±0.4. The formulation may be administered directly into an individual or may be diluted into an IV bag prior to administration.

The formulations are suitable for parenteral administration. Preferably, the formulations are administered intravenously.

The formulations may be administered parenterally, for example, as a bolus; a slow bolus over a short period of time, such as 15 minutes; a continual infusion or a continual drip. Preferably, the formulations are administered by continual infusion.

Administration by continual infusion may be at a constant rate. Alternatively, the rate of administration may be varied (not constant) over time in order to take into account loading doses prior to or at the beginning of administration and/or tapering of the infusion rate at the end of administration. Preferably, the rate of continual infusion is varied.

The aptamer formulations provided herein are administered to subjects, particularly, human subjects, in an amount effective to inhibit, reduce, block or otherwise modulate thrombin-mediated coagulation.

The formulations are used to treat, prevent or ameliorate thrombin-mediated diseases and disorders, such as coagulation disorders. The diseases and disorders to be treated, prevented or ameliorated are selected from the group consisting of: renal impairment, impaired hepatic function and heparin induced thrombocytopenia. Aptamers of the invention also provide a safe and effective modality for modulating coagulation, particularly for anticoagulation in relation to kidney dialysis and surgical procedures, such as percutaneous coronary intervention (PCI), stent placement surgery, peripheral arterial occlusion disease (PAOD), cardiopulmonary bypass procedures, coronary artery bypass graft (CABG) surgery, angioplasty, cardiovascular and peripheral vascular open and endovascular surgery, heart valve replacement surgery, and surgery to treat coronary disease and/or vascular disease in veins or arteries.

The formulations may be administered in combination with other drugs or therapies. For example, the formulations may be used in combination with other anticoagulants, such as heparin and aspirin.

The formulations may be packaged for use in a variety of pharmaceutically acceptable containers using any pharmaceutically acceptable container closure, as the formulations are compatible with PVC-containing and PVC-free containers and container closures.

The formulations may also be packaged in a kit.

In another embodiment of the invention, the use of ARC2172 in the manufacture of a medicament, pharmaceutical composition or formulation for the treatment, prevention or amelioration of a disease mediated by thrombin is provided.

In yet a further embodiment of the invention, the use of ARC2172 in the manufacture of a medicament, pharmaceutical composition or formulation for the treatment, prevention or amelioration of a disease mediated by thrombin is provided, wherein the medicament, pharmaceutical composition or formulation comprises ARC2172 and a 0.9% saline solution.

In yet a further embodiment of the invention, the use of ARC2172 in the manufacture of a medicament, pharmaceutical composition or formulation for the treatment, prevention or amelioration of a disease mediated by thrombin is provided, wherein the medicament, pharmaceutical composition or formulation is for administration to a patient in a total dosage amount of aptamer in the range of (i) 1.0-30.0 mg/kg, preferably 7.0-25.0 mg/kg, more preferably 9.0-20.0 mg/kg, even more preferably 11.0-16.0 mg/kg, and most preferably 12.0-15.0 mg/kg, or (ii) 10-6000 mg, preferably 70-5000 mg, more preferably 90-4000 mg, even more preferably 110-3200 mg, and most preferably 120-3000 mg for a 10-200 kg patient.

In another embodiment of the invention a medicament, pharmaceutical composition or formulation is provided comprising ARC2172 for use in the treatment, prevention or amelioration of a disease mediated by thrombin.

In yet a further embodiment of the invention a medicament, pharmaceutical composition or formulation is provided comprising ARC2172 and a 0.9% saline solution for use in the treatment, prevention or amelioration of a disease mediated by thrombin.

In yet a further embodiment of the invention a medicament, pharmaceutical composition or formulation is provided comprising ARC2172 in a total dosage amount in the range (i) 1.0-30.0 mg/kg, preferably 7.0-25.0 mg/kg, more preferably 9.0-20.0 mg/kg, even more preferably 11.0-16.0 mg/kg, and most preferably 12.0-15.0 mg/kg, or (ii) 10-6000 mg, preferably 70-5000 mg, more preferably 90-4000 mg, even more preferably 110-3200, and most preferably 120-3000 mg for a 10-200 kg patient, wherein the medicament, pharmaceutical composition or formulation is for use in the treatment, prevention or amelioration of a disease mediated by thrombin.

### DETAILED DESCRIPTION

The details of one or more embodiments of the invention are set forth in the accompanying description below. Although any materials and methods similar or equivalent to those described herein can be used in the practice or testing of the present invention, the preferred materials and methods are now described. Other features, objects and advantages of the invention will be apparent from the description. In the description, the singular forms also include the plural unless the context clearly dictates otherwise. Unless defined otherwise, all technical and scientific terms used herein have the same meaning as commonly understood by one of ordinary skill in the art to which this invention belongs. In the case of conflict, the present description will control.

The present invention provides pharmaceutical compositions or formulations of aptamers that bind to thrombin, referred to herein as “anti-thrombin aptamers”, and methods for using such anti-thrombin aptamers to treat thrombin-mediated diseases and disorders for decreasing or inhibiting thrombin-mediated coagulation and causing anticoagulation. The formulations comprise an anti-thrombin aptamer or a pharmaceutically acceptable salt thereof, and a pharmaceutically acceptable solvent. The formulations and dosages described herein are designed to maximize clinical efficacy in the treatment of thrombin-mediated diseases and disorders while simultaneously decreasing or minimizing adverse side effects, without the need for an antidote.

The formulations described herein are stable. The term “stable”, as used herein, means remaining in a state or condition that is suitable for administration to a patient.

The formulations are, preferably, substantially pure. As used herein, “substantially pure” means that the active ingredient (aptamer) is the predominant species present (i.e., on a molar basis it is more abundant than any other individual species in the composition), and preferably a substantially purified fraction is a composition wherein the active ingredient comprises at least about 50 percent (on a molar basis) of all macromolecular species present. Generally, a substantially pure composition will comprise more than 80% of all macromolecular species present in the composition, more preferably more than 85%, 90%, 95% and 99%. Most preferably, the active ingredient is purified to essential homogeneity (contaminant species cannot be detected in the composition by conventional detection methods) wherein the composition consists essentially of a single macromolecular species.

### Anti-Thrombin Aptamers

The formulations described herein comprise an anti-thrombin aptamer or a pharmaceutically acceptable salt thereof. The formulations may comprise any aptamer or a combination of aptamers that bind to thrombin or a variant or a fragment thereof. Preferably, the aptamer binds full length thrombin. More preferably, the aptamer binds exosite I of thrombin. The thrombin protein may be from any species, but is preferably human. The anti-thrombin aptamer preferably comprises a dissociation constant for human thrombin, or a variant thereof, of 100 nM or less, preferably 50 nM or less, more preferably 10 nM or less, even more preferably 5 nM or less, even more preferably 1 nM or less, and most preferably 500 pM or less.

Aptamers and methods for identifying an aptamer that binds to thrombin are known in the art. For example, U.S. patent application Ser. Nos. 11/990,998 and 11/443,768 (and their corresponding applications) describe such aptamers and methods in detail. For example, any of the following aptamers may be used in the formulations and methods of the invention: ARC2172 (NU172), ARC2171, ARC2170, ARC2169 and ARCminimer. Aptamers, including chemically substituted aptamers, can be synthesized using any oligonucleotide synthesis techniques known in the art, including solid phase oligonucleotide synthesis techniques (see, e.g., Froehler et al., Nucl. Acid Res., 14:5399-5467 (1986) and Froehler et al., Tet. Lett., 27:5575-5578 (1986)) and solution phase methods, such as triester synthesis methods (see, e.g., Sood et al., Nucl. Acid Res., 4:2557 (1977) and Hirose et al., Tet. Lett., 28:2449 (1978)).

The anti-thrombin aptamers used in the formulations and methods of the invention are ribonucleic acid, deoxyribonucleic acid, or mixed ribonucleic acid and deoxyribonucleic acid. Aptamers may be single stranded ribonucleic acid, deoxyribonucleic acid or mixed ribonucleic acid and deoxyribonucleic acid. In some embodiments, the aptamer comprises at least one chemical modification. In some embodiments, the chemical modification is selected from a chemical substitution of the nucleic acid at a sugar position, a chemical substitution at a phosphate position and a chemical substitution at a base position. In other embodiments, the chemical modification is selected from incorporation of a modified nucleotide; 3′ capping; conjugation to a high molecular weight, non-immunogenic compound; conjugation to a lipophilic compound; and incorporation of phosphorothioate into the phosphate backbone. In a preferred embodiment, the high molecular weight, non-immunogenic compound is polyalkylene glycol, and more preferably is polyethylene glycol (PEG).

Those of ordinary skill in the art will appreciate that the composition of an oligonucleotide can influence complement activation. For example, the number of phosphorothioate substitutions is directly related to the relative degree of complement activation. Thus, in a preferred embodiment, the anti-thrombin aptamer used in the formulations and methods provided herein has a nucleotide sequence that includes no more than four, no more than three, no more than two or no more than one phosphorothioate backbone modification. Preferably, the binding affinity for thrombin is increased relative to a second aptamer having the same nucleotide sequence but lacking the phosphorothioate backbone modification.

The anti-thrombin aptamer used in the formulations and methods described herein decrease or inhibit thrombin-mediated coagulation. In some embodiments, the ability of the aptamer to decrease or inhibit thrombin-mediated coagulation is assessed by measuring the aptamer's ability to increase or inhibit activated clotting time (ACT), prothrombin time (PT) and/or activated partial thromboplastin time (aPTT). Preferably, thrombin-mediated coagulation in the presence of an aptamer is assessed by measuring the aptamer's ability to increase ACT. In a preferred embodiment, the ability of the aptamer to decrease or inhibit coagulation is assessed by measuring ACT using a HEMOCHRON JR.® instrument, (ITC Med, Edison N.J.). In addition, the anti-thrombin aptamers have rapid and predictable onset and offset of anticoagulation, without the need for an antidote.

Preferably, the anti-thrombin aptamer binds to thrombin or a variant or a fragment thereof. More preferably, the anti-thrombin aptamer binds to exosite I of thrombin.

Preferably, the anti-thrombin aptamer acts as an antagonist to inhibit the function of thrombin. When thrombin is activated, it is responsible for coagulation.

Preferably, the anti-thrombin aptamer is ARC2172, which is also known as NU172. ARC2172, is a synthetically manufactured DNA aptamer.

ARC2172 is a DNA aptamer having the following structure:

| | |
|---|---|
| CGCCTAGGTTGGGTAGGGTGGTGGCG. | (SEQ ID NO: 1) |

ARC2172 binds human thrombin and prevents the interaction of thrombin with fibrinogen. Therefore, ARC2172 is a competitive antagonist of the thrombin/fibrinogen interaction.

ARC2172 has a molecular formula of C₂₅₆H₃₁₉N₁₀₄O₁₅₈P₂₅ (free acid form), with a molecular weight of 8,155.24 Daltons. The sodium salt of ARC2172 has the molecular formula of C₂₅₆H₂₉₄Na₂₅N₁₀₄O₁₅₈P₂₅ and corresponding molecular weight of 8704.77 Daltons.

The chemical name for the sodium salt of ARC2172 (SEQ ID NO: 1) is 2′-deoxycytidylyl-(3′→5′ O,O-phosphoryl)-2′-deoxyguanosylyl-(3′→5′ O,O-phosphoryl)-2′-deoxycytidylyl-(3′→5′ O,O-phosphoryl)-2′-deoxycytidylyl-(3′→5′ O,O-phosphoryl)-2′-deoxythymidylyl-(3′→5′ O,O-phosphoryl)-2′-deoxyadenosylyl-(3′→5′ O,O-phosphoryl)-2′-deoxyguanosylyl-(3′→5′ O,O-phosphoryl)-2′-deoxyguanosylyl-(3′→5′ O,O-phosphoryl)-2′-deoxythymidylyl-(3′→5′ O,O-phosphoryl)-2′-deoxythymidylyl-(3′→5′ O,O-phosphoryl)-2′-deoxyguanosylyl-(3′→5′ O,O-phosphoryl)-2′-deoxyguanosylyl-(3′→5′ O,O-phosphoryl)-2′-deoxyguanosylyl-(3′→5′ O,O-phosphoryl)-2′-deoxythymidylyl-(3′→5′ O,O-phosphoryl)-2′-deoxyadenosylyl-(3′→5′ O,O-phosphoryl)-2′-deoxyguanosylyl-(3′→5′ O,O-phosphoryl)-2′-deoxyguanosylyl-(3′→5′ O,O-phosphoryl)-2′-deoxyguanosylyl-(3′→5′ O,O-phosphoryl)-2′-deoxythymidylyl-(3′→5′ O,O-phosphoryl)-2′-deoxyguanosylyl-(3′→5′ O,O-phosphoryl)-2′-deoxyguanosylyl-(3′→5′ O,O-phosphoryl)-2′-deoxythymidylyl-(3′→5′ O,O-phosphoryl)-2′-deoxyguanosylyl-(3′→5′ O,O-phosphoryl)-2′-deoxyguanosylyl-(3′→5′ O,O-phosphoryl)-2′-deoxycytidylyl-(3′→5′ O,O-phosphoryl)-2′-deoxyguanosine, 25-sodium salt. ARC2172 is soluble in water.

The formulations described herein may use ARC2172 and/or other aptamers specifically capable of binding and modulating, e.g., antagonizing, full length thrombin and/or the exosite I domain of thrombin.

Examples of such other aptamers include, but are not limited to, ARC2171, ARC2170, ARC2169 and ARCminimer.

ARC2171 is an aptamer having the following structure:

| | |
|---|---|
| CTGCCTAGGTTGGGTAGGGTGGTGGCAG. | (SEQ ID NO: 2) |

ARC2170 is an aptamer having the following structure:

| | |
|---|---|
| GCTGCCTAGGTTGGGTAGGGTGGTGGCAGC. | (SEQ ID NO: 3) |

ARC2169 is an aptamer having the following structure:

| | |
|---|---|
| ACTGCCTAGGTTGGGTAGGGTGGTGGCAGT. | (SEQ ID NO: 4) |

ARCminimer is an aptamer having the following structure:

| | |
|---|---|
| CCTAGGTTGGGTAGGGTGGTGG. | (SEQ ID NO: 5) |

As stated previously, the formulations may comprise an anti-thrombin aptamer or its pharmaceutically acceptable salt. As used herein, the term “pharmaceutically acceptable salt” refers to salt forms of the active compound that are prepared with counter ions that are non-toxic under the conditions of use and are compatible with a stable formulation. Examples of pharmaceutically acceptable salts of anti-thrombin aptamers include sodium salts, hydrochlorides, sulfates, phosphates, acetates, fumarates, maleates and tartrates.

The formulations may comprise any amount of an anti-thrombin aptamer. Typically, the formulations comprise 10-100 mg/ml of anti-thrombin aptamer or a pharmaceutically acceptable salt thereof. For example, suitable concentrations of anti-thrombin aptamer or a pharmaceutically acceptable salt thereof include, but are not limited to, 10-100 mg/ml or any 0.1 mg/ml increment thereof. Preferably, formulations comprise 10-50 mg/ml of anti-thrombin aptamer or a pharmaceutically acceptable salt thereof. More preferably, formulations comprise 10-30 mg/ml of anti-thrombin aptamer or a pharmaceutically acceptable salt thereof. Even more preferably, formulations comprise 15-30 mg/ml of anti-thrombin aptamer or a pharmaceutically acceptable salt thereof. Most preferably, formulations comprise 15 mg/ml of anti-thrombin aptamer or a pharmaceutically acceptable salt thereof.

### Pharmaceutically Acceptable Solvent

The formulations also comprise a pharmaceutically acceptable solvent. The term “pharmaceutically acceptable solvent”, as used herein, means being compatible with the other ingredients of the formulation and not deleterious to the recipient thereof. Pharmaceutically acceptable solvents are well known in the art. Examples of pharmaceutically acceptable solvents can be found, for example, in Goodman and Gillmans, The Pharmacological Basis of Therapeutics, latest edition. Preferably, the pharmaceutically acceptable solvent is selected from the group consisting of 0.9% saline (physiological saline or sterile isotonic saline solution) or phosphate buffered saline. Most preferably, the pharmaceutically acceptable solvent is 0.9% saline.

The formulations may comprise any amount of pharmaceutically acceptable solvent.

### Optional Components

The formulations of the invention only require an anti-thrombin aptamer or a pharmaceutically acceptable salt thereof, and a pharmaceutically acceptable solvent. However, various embodiments of the formulations may, optionally, include one or more of the following: buffer, pH adjuster, tonicity agent, cosolvent or pharmaceutically acceptable carrier.

In some embodiments, the formulations may further comprise a buffer. A buffer is any substance that, when added to a solution, is capable of neutralizing both acids and bases without appreciably changing acidity or alkalinity of the solution. Examples of buffers include, but are not limited to, pharmaceutically acceptable salts and acids of acetate, glutamate, citrate, tartrate, benzoate, lactate, histidine or other amino acids, gluconate, phosphate, malate, succinate, formate, propionate and carbonate.

In some embodiments, the formulations may further comprise a pH adjuster. A pH adjuster is used to adjust the pH of the formulation. Suitable pH adjusters typically include at least an acid or a salt thereof and/or a base or a salt thereof. Acids and bases can be added on an as needed basis in order to achieve a desired pH. For example, if the pH is greater than the desired pH, an acid may be used to lower the pH to the desired pH. Examples of acids include, but are not limited to, hydrochloric acid, phosphoric acid, citric acid, ascorbic acid, acetic acid, sulphuric acid, carbonic acid and nitric acid. By way of another example, if the pH is less than the desired pH, a base can be used to adjust the pH to the desired pH. Examples of bases include, but are not limited to, sodium hydroxide, potassium hydroxide, calcium hydroxide, sodium carbonate, sodium citrate, sodium acetate and magnesium hydroxide.

In some embodiments, the formulations may further comprise a tonicity agent. Tonicity agents are used to adjust the osmolality of the formulations in order to bring them closer to the osmotic pressure of body fluids, such as blood or plasma. Examples of tonicity agents include, but are not limited to, anhydrous or hydrous forms of sodium chloride, dextrose, sucrose, xylitol, fructose, glycerol, sorbitol, mannitol, potassium chloride, mannose, calcium chloride, magnesium chloride and other inorganic salts.

In some embodiments, the formulations may further comprise a cosolvent. A cosolvent is a solvent that is added to the aqueous formulation in a weight amount that is less than that of water and assists in the solubilization of the anti-thrombin aptamer. Examples of cosolvents include, but are not limited to, glycols, ethanol and polyhydric alcohols.

In some embodiments, the formulations may further comprise a pharmaceutically acceptable carrier. The term “pharmaceutically acceptable carrier”, as used herein, means being compatible with the other ingredients of the formulation and not deleterious to the recipient thereof. Pharmaceutically acceptable carriers are well known in the art. Examples of pharmaceutically acceptable carriers can be found, for example, in Goodman and Gillmans, The Pharmacological Basis of Therapeutics, latest edition.

### Dosing

The total dose of anti-thrombin aptamer is administered so as to achieve a steady state blood concentration equal to at least the EC₉₀, and preferably of 10-35 μg/ml. The steady state blood concentration can be any concentration between, and including, 10 μg/ml and 35 μg/ml in increments of 0.1 μg/ml. For example, acceptable steady state blood concentrations include, but are not limited to, 10 μg/ml, 11 μg/ml, 12 μg/ml, 13 μg/ml, 14 μg/ml, 15 μg/ml, 16 μg/ml, 17 μg/ml, 18 μg/ml, 19 μg/ml, 20 μg/ml, 21 μg/ml, 22 μg/ml, 23 μg/ml, 24 μg/ml, 25 μg/ml, 26 μg/ml, 27 μg/ml, 28 μg/ml, 29 μg/ml, 30 μg/ml, 31 μg/ml, 32 μg/ml, 33 μg/ml, 34 μg/ml and 35 μg/ml. Preferably, the total dose achieves a steady state blood concentration of 15-25 μg/ml. More preferably, the total dose achieves a steady state blood concentration of 18-22 μg/ml. Most preferably, the total dose achieves a steady state blood concentration of 20 μg/ml.

The total dose of anti-thrombin aptamer is administered in mg per kg (mg/kg) of body weight. The total dose of anti-thrombin aptamer is 1-30 mg/kg. The total dose can be any dose between, and including, 1.0 mg/kg and 30.0 mg/kg in increments of 0.1 mg/kg. For example, acceptable dosage ranges include, but are not limited to, 1.0-2.0 mg/kg, 1.5-2.5 mg/kg, 2-3 mg/kg, 2.5-3.5 mg/kg, 3-4 mg/kg, 3.5-4.5 mg/kg, 4-5 mg/kg, 4.5-5.5 mg/kg, 5-6 mg/kg, 5.5-6.5 mg/kg, 6-7 mg/kg, 6.5-7.5 mg/kg, 7-8 mg/kg, 7.5-8.5 mg/kg, 8-9 mg/kg, 8.5-9.5 mg/kg, 9-10 mg/kg, 9.5-10.5 mg/kg, 10-11 mg/kg, 10.5-11.5 mg/kg, 11.0-12.0 mg/kg, 11.5-12.5 mg/kg, 12-13 mg/kg, 12.5-13.5 mg/kg, 13-14 mg/kg, 13.5-14.5 mg/kg, 14-15 mg/kg, 14.5-15.5 mg/kg, 15-16 mg/kg, 15.5-16.5 mg/kg, 16-17 mg/kg, 16.5-17.5 mg/kg, 17-18 mg/kg, 17.5-18.5 mg/kg, 18-19 mg/kg, 18.5-19.5 mg/kg, 19-20 mg/kg; 19.5-20.5 mg/kg, 20-21 mg/kg, 20.5-21.5 mg/kg, 21-22 mg/kg, 21.5-22.5 mg/kg, 22-23 mg/kg, 22.5-23.5 mg/kg, 23-24 mg/kg, 23.5-24.5 mg/kg, 24-25 mg/kg, 24.5-25.5 mg/kg, 25-26 mg/kg, 25.5-26.5 mg/kg, 26-27 mg/kg, 26.5-27.5 mg/kg, 27-28 mg/kg, 27.5-28.5 mg/kg, 28-29 mg/kg, 28.5-29.5 mg/kg, 29-30 mg/kg, and 29.5-30 mg/kg. This total dosage may be administered in a single dose, multiple doses, as a continual dose or a combination thereof.

Preferably, a total dose of anti-thrombin aptamer of 7-25 mg/kg is administered. More preferably, a total dose of 9-20 mg/kg is administered. Even more preferably, a total dose of 11-16 mg/kg is administered. Most preferably, a total dose of 12-15 mg/kg is administered. This total dosage may be administered in a single dose, multiple doses, as a continual dose or a combination thereof. For example, a loading dose or doses may be administered followed by a maintenance dose or doses. The exact dose, however, is determined by the physician and is dependent upon many factors, such as the age, weight, condition and response of the patient. To achieve the desired blood concentration level, any total dose of 1.0-30.0 mg/kg can be used.

For example, for a body weight of 10-200 kg, the total dosage level (1-30 mg/kg) of anti-thrombin aptamer is 10-6000 mg. For an average body weight of 70 kg, the dosage level is 70-2100 mg. The total dose may be any one of 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995, 1000, 1005, 1010, 1015, 1020, 1025, 1030, 1035, 1040, 1045, 1050, 1055, 1060, 1065, 1070, 1075, 1080, 1085, 1090, 1095, 1100, 1105, 1110, 1115, 1120, 1125, 1130, 1135, 1140, 1145, 1150, 1155, 1160, 1165, 1170, 1175, 1180, 1185, 1190, 1195, 1200, 1205, 1210, 1215, 1220, 1225, 1230, 1235, 1240, 1245, 1250, 1255, 1260, 1265, 1270, 1275, 1280, 1285, 1290, 1295, 1300, 1305, 1310, 1315, 1320, 1325, 1330, 1335, 1340, 1345, 1350, 1355, 1360, 1365, 1370, 1375, 1380, 1385, 1390, 1395, 1400, 1405, 1410, 1415, 1420, 1425, 1430, 1435, 1440, 1445, 1450, 1455, 1460, 1465, 1470, 1475, 1480, 1485, 1490, 1495, 1500, 1505, 1510, 1515, 1520, 1525, 1530, 1535, 1540, 1545, 1550, 1555, 1560, 1565, 1570, 1575, 1580, 1585, 1590, 1595, 1600, 1605, 1610, 1615, 1620, 1625, 1630, 1635, 1640, 1645, 1650, 1655, 1660, 1665, 1670, 1675, 1680, 1685, 1690, 1695, 1700, 1705, 1710, 1715, 1720, 1725, 1730, 1735, 1740, 1745, 1750, 1755, 1760, 1765, 1770, 1775, 1780, 1785, 1790, 1795, 1800, 1805, 1810, 1815, 1820, 1825, 1830, 1835, 1840, 1845, 1850, 1855, 1860, 1865, 1870, 1875, 1880, 1885, 1890, 1895, 1900, 1905, 1910, 1915, 1920, 1925, 1930, 1935, 1940, 1945, 1950, 1955, 1960, 1965, 1970, 1975, 1980, 1985, 1990, 1995, 2000, 2010, 2015, 2020, 2025, 2030, 2035, 2040, 2045, 2050, 2055, 2060, 2065, 2070, 2075, 2080, 2085, 2090, 2095, 2100, 2105, 2110, 2115, 2120, 2125, 2130, 2135, 2140, 2145, 2150, 2155, 2160, 2165, 2170, 2175, 2180, 2185, 2190, 2195, 2200, 2205, 2210, 2215, 2220, 2225, 2230, 2235, 2240, 2245, 2250, 2255, 2260, 2265, 2270, 2275, 2280, 2285, 2290, 2295, 2300, 2305, 2310, 2315, 2320, 2325, 2330, 2335, 2340, 2345, 2350, 2355, 2360, 2365, 2370, 2375, 2380, 2385, 2390, 2395, 2400, 2405, 2410, 2415, 2420, 2425, 2430, 2435, 2440, 2445, 2450, 2455, 2460, 2465, 2470, 2475, 2480, 2485, 2490, 2495, 2500, 2505, 2510, 2515, 2520, 2525, 2530, 2535, 2540, 2545, 2550, 2555, 2560, 2565, 2570, 2575, 2580, 2585, 2590, 2595, 2600, 2605, 2610, 2615, 2620, 2625, 2630, 2635, 2640, 2645, 2650, 2655, 2660, 2665, 2670, 2675, 2680, 2685, 2690, 2695, 2700, 2705, 2710, 2715, 2720, 2725, 2730, 2735, 2740, 2745, 2750, 2755, 2760, 2765, 2770, 2775, 2780, 2785, 2790, 2795, 2800, 2805, 2810, 2815, 2820, 2825, 2830, 2835, 2840, 2845, 2850, 2855, 2860, 2865, 2870, 2875, 2880, 2885, 2890, 2895, 2900, 2905, 2910, 2915, 2920, 2925, 2930, 2935, 2940, 2945, 2950, 2955, 2960, 2965, 2970, 2975, 2980, 2985, 2990, 2995, 3000, 3010, 3015, 3020, 3025, 3030, 3035, 3040, 3045, 3050, 3055, 3060, 3065, 3070, 3075, 3080, 3085, 3090, 3095, 3100, 3105, 3110, 3115, 3120, 3125, 3130, 3135, 3140, 3145, 3150, 3155, 3160, 3165, 3170, 3175, 3180, 3185, 3190, 3195, 3200, 3205, 3210, 3215, 3220, 3225, 3230, 3235, 3240, 3245, 3250, 3255, 3260, 3265, 3270, 3275, 3280, 3285, 3290, 3295, 3300, 3305, 3310, 3315, 3320, 3325, 3330, 3335, 3340, 3345, 3350, 3355, 3360, 3365, 3370, 3375, 3380, 3385, 3390, 3395, 3400, 3405, 3410, 3415, 3420, 3425, 3430, 3435, 3440, 3445, 3450, 3455, 3460, 3465, 3470, 3475, 3480, 3485, 3490, 3495, 3500, 3505, 3510, 3515, 3520, 3525, 3530, 3535, 3540, 3545, 3550, 3555, 3560, 3565, 3570, 3575, 3580, 3585, 3590, 3595, 3600, 3605, 3610, 3615, 3620, 3625, 3630, 3635, 3640, 3645, 3650, 3655, 3660, 3665, 3670, 3675, 3680, 3685, 3690, 3695, 3700, 3705, 3710, 3715, 3720, 3725, 3730, 3735, 3740, 3745, 3750, 3755, 3760, 3765, 3770, 3775, 3780, 3785, 3790, 3795, 3800, 3805, 3810, 3815, 3820, 3825, 3830, 3835, 3840, 3845, 3850, 3855, 3860, 3865, 3870, 3875, 3880, 3885, 3890, 3895, 3900, 3905, 3910, 3915, 3920, 3925, 3930, 3935, 3940, 3945, 3950, 3955, 3960, 3965, 3970, 3975, 3980, 3985, 3990, 3995, 4000, 4010, 4015, 4020, 4025, 4030, 4035, 4040, 4045, 4050, 4055, 4060, 4065, 4070, 4075, 4080, 4085, 4090, 4095, 4100, 4105, 4110, 4115, 4120, 4125, 4130, 4135, 4140, 4145, 4150, 4155, 4160, 4165, 4170, 4175, 4180, 4185, 4190, 4195, 4200, 4205, 4210, 4215, 4220, 4225, 4230, 4235, 4240, 4245, 4250, 4255, 4260, 4265, 4270, 4275, 4280, 4285, 4290, 4295, 4300, 4305, 4310, 4315, 4320, 4325, 4330, 4335, 4340, 4345, 4350, 4355, 4360, 4365, 4370, 4375, 4380, 4385, 4390, 4395, 4400, 4405, 4410, 4415, 4420, 4425, 4430, 4435, 4440, 4445, 4450, 4455, 4460, 4465, 4470, 4475, 4480, 4485, 4490, 4495, 4500, 4505, 4510, 4515, 4520, 4525, 4530, 4535, 4540, 4545, 4550, 4555, 4560, 4565, 4570, 4575, 4580, 4585, 4590, 4595, 4600, 4605, 4610, 4615, 4620, 4625, 4630, 4635, 4640, 4645, 4650, 4655, 4660, 4665, 4670, 4675, 4680, 4685, 4690, 4695, 4700, 4705, 4710, 4715, 4720, 4725, 4730, 4735, 4740, 4745, 4750, 4755, 4760, 4765, 4770, 4775, 4780, 4785, 4790, 4795, 4800, 4805, 4810, 4815, 4820, 4825, 4830, 4835, 4840, 4845, 4850, 4855, 4860, 4865, 4870, 4875, 4880, 4885, 4890, 4895, 4900, 4905, 4910, 4915, 4920, 4925, 4930, 4935, 4940, 4945, 4950, 4955, 4960, 4965, 4970, 4975, 4980, 4985, 4990, 4995, 5000, 5005, 5010, 5015, 5020, 5025, 5030, 5035, 5040, 5045, 5050, 5055, 5060, 5065, 5070, 5075, 5080, 5085, 5090, 5095, 5100, 5105, 5110, 5115, 5120, 5125, 5130, 5135, 5140, 5145, 5150, 5155, 5160, 5165, 5170, 5175, 5180, 5185, 5190, 5195, 5200, 5205, 5210, 5215, 5220, 5225, 5230, 5235, 5240, 5245, 5250, 5255, 5260, 5265, 5270, 5275, 5280, 5285, 5290, 5295, 5300, 5305, 5310, 5315, 5320, 5325, 5330, 5335, 5340, 5345, 5350, 5355, 5360, 5365, 5370, 5375, 5380, 5385, 5390, 5395, 5400, 5405, 5410, 5415, 5420, 5425, 5430, 5435, 5440, 5445, 5450, 5455, 5460, 5465, 5470, 5475, 5480, 5485, 5490, 5495, 5500, 5505, 5510, 5515, 5520, 5525, 5530, 5535, 5540, 5545, 5550, 5555, 5560, 5565, 5570, 5575, 5580, 5585, 5590, 5595, 5600, 5605, 5610, 5615, 5620, 5625, 5630, 5635, 5640, 5645, 5650, 5655, 5660, 5665, 5670, 5675, 5680, 5685, 5690, 5695, 5700, 5705, 5710, 5715, 5720, 5725, 5730, 5735, 5740, 5745, 5750, 5755, 5760, 5765, 5770, 5775, 5780, 5785, 5790, 5795, 5800, 5805, 5810, 5815, 5820, 5825, 5830, 5835, 5840, 5845, 5850, 5855, 5860, 5865, 5870, 5875, 5880, 5885, 5890, 5895, 5900, 5905, 5910, 5915, 5920, 5925, 5930, 5935, 5940, 5945, 5950, 5955, 5960, 5965, 5970, 5975, 5980, 5985, 5990, 5995 and 6000 mg.

For example, for a body weight of 10-50 kg, the total dosage level (1-30 mg/kg) of anti-thrombin aptamer is 10-1500 mg. The total dose may be any one of 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995, 1000, 1005, 1010, 1015, 1020, 1025, 1030, 1035, 1040, 1045, 1050, 1055, 1060, 1065, 1070, 1075, 1080, 1085, 1090, 1095, 1100, 1105, 1110, 1115, 1120, 1125, 1130, 1135, 1140, 1145, 1150, 1155, 1160, 1165, 1170, 1175, 1180, 1185, 1190, 1195, 1200, 1205, 1210, 1215, 1220, 1225, 1230, 1235, 1240, 1245, 1250, 1255, 1260, 1265, 1270, 1275, 1280, 1285, 1290, 1295, 1300, 1305, 1310, 1315, 1320, 1325, 1330, 1335, 1340, 1345, 1350, 1355, 1360, 1365, 1370, 1375, 1380, 1385, 1390, 1395, 1400, 1405, 1410, 1415, 1420, 1425, 1430, 1435, 1440, 1445, 1450, 1455, 1460, 1465, 1470, 1475, 1480, 1485, 1490, 1495 and 1500 mg.

For example, for a body weight of 50-100 kg, the total dosage level (1-30 mg/kg) of anti-thrombin aptamer is 50-3000 mg. The total dose may be any one of 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995, 1000, 1005, 1010, 1015, 1020, 1025, 1030, 1035, 1040, 1045, 1050, 1055, 1060, 1065, 1070, 1075, 1080, 1085, 1090, 1095, 1100, 1105, 1110, 1115, 1120, 1125, 1130, 1135, 1140, 1145, 1150, 1155, 1160, 1165, 1170, 1175, 1180, 1185, 1190, 1195, 1200, 1205, 1210, 1215, 1220, 1225, 1230, 1235, 1240, 1245, 1250, 1255, 1260, 1265, 1270, 1275, 1280, 1285, 1290, 1295, 1300, 1305, 1310, 1315, 1320, 1325, 1330, 1335, 1340, 1345, 1350, 1355, 1360, 1365, 1370, 1375, 1380, 1385, 1390, 1395, 1400, 1405, 1410, 1415, 1420, 1425, 1430, 1435, 1440, 1445, 1450, 1455, 1460, 1465, 1470, 1475, 1480, 1485, 1490, 1495, 1500, 1505, 1510, 1515, 1520, 1525, 1530, 1535, 1540, 1545, 1550, 1555, 1560, 1565, 1570, 1575, 1580, 1585, 1590, 1595, 1600, 1605, 1610, 1615, 1620, 1625, 1630, 1635, 1640, 1645, 1650, 1655, 1660, 1665, 1670, 1675, 1680, 1685, 1690, 1695, 1700, 1705, 1710, 1715, 1720, 1725, 1730, 1735, 1740, 1745, 1750, 1755, 1760, 1765, 1770, 1775, 1780, 1785, 1790, 1795, 1800, 1805, 1810, 1815, 1820, 1825, 1830, 1835, 1840, 1845, 1850, 1855, 1860, 1865, 1870, 1875, 1880, 1885, 1890, 1895, 1900, 1905, 1910, 1915, 1920, 1925, 1930, 1935, 1940, 1945, 1950, 1955, 1960, 1965, 1970, 1975, 1980, 1985, 1990, 1995, 2000, 2010, 2015, 2020, 2025, 2030, 2035, 2040, 2045, 2050, 2055, 2060, 2065, 2070, 2075, 2080, 2085, 2090, 2095, 2100, 2105, 2110, 2115, 2120, 2125, 2130, 2135, 2140, 2145, 2150, 2155, 2160, 2165, 2170, 2175, 2180, 2185, 2190, 2195, 2200, 2205, 2210, 2215, 2220, 2225, 2230, 2235, 2240, 2245, 2250, 2255, 2260, 2265, 2270, 2275, 2280, 2285, 2290, 2295, 2300, 2305, 2310, 2315, 2320, 2325, 2330, 2335, 2340, 2345, 2350, 2355, 2360, 2365, 2370, 2375, 2380, 2385, 2390, 2395, 2400, 2405, 2410, 2415, 2420, 2425, 2430, 2435, 2440, 2445, 2450, 2455, 2460, 2465, 2470, 2475, 2480, 2485, 2490, 2495, 2500, 2505, 2510, 2515, 2520, 2525, 2530, 2535, 2540, 2545, 2550, 2555, 2560, 2565, 2570, 2575, 2580, 2585, 2590, 2595, 2600, 2605, 2610, 2615, 2620, 2625, 2630, 2635, 2640, 2645, 2650, 2655, 2660, 2665, 2670, 2675, 2680, 2685, 2690, 2695, 2700, 2705, 2710, 2715, 2720, 2725, 2730, 2735, 2740, 2745, 2750, 2755, 2760, 2765, 2770, 2775, 2780, 2785, 2790, 2795, 2800, 2805, 2810, 2815, 2820, 2825, 2830, 2835, 2840, 2845, 2850, 2855, 2860, 2865, 2870, 2875, 2880, 2885, 2890, 2895, 2900, 2905, 2910, 2915, 2920, 2925, 2930, 2935, 2940, 2945, 2950, 2955, 2960, 2965, 2970, 2975, 2980, 2985, 2990, 2995 and 3000 mg.

For example, for a body weight of 100-150 kg, the total dosage level (1-30 mg/kg) of anti-thrombin aptamer is 100-4500 mg. The total dose may be any one of 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995, 1000, 1005, 1010, 1015, 1020, 1025, 1030, 1035, 1040, 1045, 1050, 1055, 1060, 1065, 1070, 1075, 1080, 1085, 1090, 1095, 1100, 1105, 1110, 1115, 1120, 1125, 1130, 1135, 1140, 1145, 1150, 1155, 1160, 1165, 1170, 1175, 1180, 1185, 1190, 1195, 1200, 1205, 1210, 1215, 1220, 1225, 1230, 1235, 1240, 1245, 1250, 1255, 1260, 1265, 1270, 1275, 1280, 1285, 1290, 1295, 1300, 1305, 1310, 1315, 1320, 1325, 1330, 1335, 1340, 1345, 1350, 1355, 1360, 1365, 1370, 1375, 1380, 1385, 1390, 1395, 1400, 1405, 1410, 1415, 1420, 1425, 1430, 1435, 1440, 1445, 1450, 1455, 1460, 1465, 1470, 1475, 1480, 1485, 1490, 1495, 1500, 1505, 1510, 1515, 1520, 1525, 1530, 1535, 1540, 1545, 1550, 1555, 1560, 1565, 1570, 1575, 1580, 1585, 1590, 1595, 1600, 1605, 1610, 1615, 1620, 1625, 1630, 1635, 1640, 1645, 1650, 1655, 1660, 1665, 1670, 1675, 1680, 1685, 1690, 1695, 1700, 1705, 1710, 1715, 1720, 1725, 1730, 1735, 1740, 1745, 1750, 1755, 1760, 1765, 1770, 1775, 1780, 1785, 1790, 1795, 1800, 1805, 1810, 1815, 1820, 1825, 1830, 1835, 1840, 1845, 1850, 1855, 1860, 1865, 1870, 1875, 1880, 1885, 1890, 1895, 1900, 1905, 1910, 1915, 1920, 1925, 1930, 1935, 1940, 1945, 1950, 1955, 1960, 1965, 1970, 1975, 1980, 1985, 1990, 1995, 2000, 2010, 2015, 2020, 2025, 2030, 2035, 2040, 2045, 2050, 2055, 2060, 2065, 2070, 2075, 2080, 2085, 2090, 2095, 2100, 2105, 2110, 2115, 2120, 2125, 2130, 2135, 2140, 2145, 2150, 2155, 2160, 2165, 2170, 2175, 2180, 2185, 2190, 2195, 2200, 2205, 2210, 2215, 2220, 2225, 2230, 2235, 2240, 2245, 2250, 2255, 2260, 2265, 2270, 2275, 2280, 2285, 2290, 2295, 2300, 2305, 2310, 2315, 2320, 2325, 2330, 2335, 2340, 2345, 2350, 2355, 2360, 2365, 2370, 2375, 2380, 2385, 2390, 2395, 2400, 2405, 2410, 2415, 2420, 2425, 2430, 2435, 2440, 2445, 2450, 2455, 2460, 2465, 2470, 2475, 2480, 2485, 2490, 2495, 2500, 2505, 2510, 2515, 2520, 2525, 2530, 2535, 2540, 2545, 2550, 2555, 2560, 2565, 2570, 2575, 2580, 2585, 2590, 2595, 2600, 2605, 2610, 2615, 2620, 2625, 2630, 2635, 2640, 2645, 2650, 2655, 2660, 2665, 2670, 2675, 2680, 2685, 2690, 2695, 2700, 2705, 2710, 2715, 2720, 2725, 2730, 2735, 2740, 2745, 2750, 2755, 2760, 2765, 2770, 2775, 2780, 2785, 2790, 2795, 2800, 2805, 2810, 2815, 2820, 2825, 2830, 2835, 2840, 2845, 2850, 2855, 2860, 2865, 2870, 2875, 2880, 2885, 2890, 2895, 2900, 2905, 2910, 2915, 2920, 2925, 2930, 2935, 2940, 2945, 2950, 2955, 2960, 2965, 2970, 2975, 2980, 2985, 2990, 2995, 3000, 3010, 3015, 3020, 3025, 3030, 3035, 3040, 3045, 3050, 3055, 3060, 3065, 3070, 3075, 3080, 3085, 3090, 3095, 3100, 3105, 3110, 3115, 3120, 3125, 3130, 3135, 3140, 3145, 3150, 3155, 3160, 3165, 3170, 3175, 3180, 3185, 3190, 3195, 3200, 3205, 3210, 3215, 3220, 3225, 3230, 3235, 3240, 3245, 3250, 3255, 3260, 3265, 3270, 3275, 3280, 3285, 3290, 3295, 3300, 3305, 3310, 3315, 3320, 3325, 3330, 3335, 3340, 3345, 3350, 3355, 3360, 3365, 3370, 3375, 3380, 3385, 3390, 3395, 3400, 3405, 3410, 3415, 3420, 3425, 3430, 3435, 3440, 3445, 3450, 3455, 3460, 3465, 3470, 3475, 3480, 3485, 3490, 3495, 3500, 3505, 3510, 3515, 3520, 3525, 3530, 3535, 3540, 3545, 3550, 3555, 3560, 3565, 3570, 3575, 3580, 3585, 3590, 3595, 3600, 3605, 3610, 3615, 3620, 3625, 3630, 3635, 3640, 3645, 3650, 3655, 3660, 3665, 3670, 3675, 3680, 3685, 3690, 3695, 3700, 3705, 3710, 3715, 3720, 3725, 3730, 3735, 3740, 3745, 3750, 3755, 3760, 3765, 3770, 3775, 3780, 3785, 3790, 3795, 3800, 3805, 3810, 3815, 3820, 3825, 3830, 3835, 3840, 3845, 3850, 3855, 3860, 3865, 3870, 3875, 3880, 3885, 3890, 3895, 3900, 3905, 3910, 3915, 3920, 3925, 3930, 3935, 3940, 3945, 3950, 3955, 3960, 3965, 3970, 3975, 3980, 3985, 3990, 3995, 4000, 4010, 4015, 4020, 4025, 4030, 4035, 4040, 4045, 4050, 4055, 4060, 4065, 4070, 4075, 4080, 4085, 4090, 4095, 4100, 4105, 4110, 4115, 4120, 4125, 4130, 4135, 4140, 4145, 4150, 4155, 4160, 4165, 4170, 4175, 4180, 4185, 4190, 4195, 4200, 4205, 4210, 4215, 4220, 4225, 4230, 4235, 4240, 4245, 4250, 4255, 4260, 4265, 4270, 4275, 4280, 4285, 4290, 4295, 4300, 4305, 4310, 4315, 4320, 4325, 4330, 4335, 4340, 4345, 4350, 4355, 4360, 4365, 4370, 4375, 4380, 4385, 4390, 4395, 4400, 4405, 4410, 4415, 4420, 4425, 4430, 4435, 4440, 4445, 4450, 4455, 4460, 4465, 4470, 4475, 4480, 4485, 4490, 4495 and 4500 mg.

For example, for a body weight of 150-200 kg, the total dosage level (1-30 mg/kg) of anti-thrombin aptamer is 150-6000 mg. The total dose may be any one of 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995, 1000, 1005, 1010, 1015, 1020, 1025, 1030, 1035, 1040, 1045, 1050, 1055, 1060, 1065, 1070, 1075, 1080, 1085, 1090, 1095, 1100, 1105, 1110, 1115, 1120, 1125, 1130, 1135, 1140, 1145, 1150, 1155, 1160, 1165, 1170, 1175, 1180, 1185, 1190, 1195, 1200, 1205, 1210, 1215, 1220, 1225, 1230, 1235, 1240, 1245, 1250, 1255, 1260, 1265, 1270, 1275, 1280, 1285, 1290, 1295, 1300, 1305, 1310, 1315, 1320, 1325, 1330, 1335, 1340, 1345, 1350, 1355, 1360, 1365, 1370, 1375, 1380, 1385, 1390, 1395, 1400, 1405, 1410, 1415, 1420, 1425, 1430, 1435, 1440, 1445, 1450, 1455, 1460, 1465, 1470, 1475, 1480, 1485, 1490, 1495, 1500, 1505, 1510, 1515, 1520, 1525, 1530, 1535, 1540, 1545, 1550, 1555, 1560, 1565, 1570, 1575, 1580, 1585, 1590, 1595, 1600, 1605, 1610, 1615, 1620, 1625, 1630, 1635, 1640, 1645, 1650, 1655, 1660, 1665, 1670, 1675, 1680, 1685, 1690, 1695, 1700, 1705, 1710, 1715, 1720, 1725, 1730, 1735, 1740, 1745, 1750, 1755, 1760, 1765, 1770, 1775, 1780, 1785, 1790, 1795, 1800, 1805, 1810, 1815, 1820, 1825, 1830, 1835, 1840, 1845, 1850, 1855, 1860, 1865, 1870, 1875, 1880, 1885, 1890, 1895, 1900, 1905, 1910, 1915, 1920, 1925, 1930, 1935, 1940, 1945, 1950, 1955, 1960, 1965, 1970, 1975, 1980, 1985, 1990, 1995, 2000, 2010, 2015, 2020, 2025, 2030, 2035, 2040, 2045, 2050, 2055, 2060, 2065, 2070, 2075, 2080, 2085, 2090, 2095, 2100, 2105, 2110, 2115, 2120, 2125, 2130, 2135, 2140, 2145, 2150, 2155, 2160, 2165, 2170, 2175, 2180, 2185, 2190, 2195, 2200, 2205, 2210, 2215, 2220, 2225, 2230, 2235, 2240, 2245, 2250, 2255, 2260, 2265, 2270, 2275, 2280, 2285, 2290, 2295, 2300, 2305, 2310, 2315, 2320, 2325, 2330, 2335, 2340, 2345, 2350, 2355, 2360, 2365, 2370, 2375, 2380, 2385, 2390, 2395, 2400, 2405, 2410, 2415, 2420, 2425, 2430, 2435, 2440, 2445, 2450, 2455, 2460, 2465, 2470, 2475, 2480, 2485, 2490, 2495, 2500, 2505, 2510, 2515, 2520, 2525, 2530, 2535, 2540, 2545, 2550, 2555, 2560, 2565, 2570, 2575, 2580, 2585, 2590, 2595, 2600, 2605, 2610, 2615, 2620, 2625, 2630, 2635, 2640, 2645, 2650, 2655, 2660, 2665, 2670, 2675, 2680, 2685, 2690, 2695, 2700, 2705, 2710, 2715, 2720, 2725, 2730, 2735, 2740, 2745, 2750, 2755, 2760, 2765, 2770, 2775, 2780, 2785, 2790, 2795, 2800, 2805, 2810, 2815, 2820, 2825, 2830, 2835, 2840, 2845, 2850, 2855, 2860, 2865, 2870, 2875, 2880, 2885, 2890, 2895, 2900, 2905, 2910, 2915, 2920, 2925, 2930, 2935, 2940, 2945, 2950, 2955, 2960, 2965, 2970, 2975, 2980, 2985, 2990, 2995, 3000, 3010, 3015, 3020, 3025, 3030, 3035, 3040, 3045, 3050, 3055, 3060, 3065, 3070, 3075, 3080, 3085, 3090, 3095, 3100, 3105, 3110, 3115, 3120, 3125, 3130, 3135, 3140, 3145, 3150, 3155, 3160, 3165, 3170, 3175, 3180, 3185, 3190, 3195, 3200, 3205, 3210, 3215, 3220, 3225, 3230, 3235, 3240, 3245, 3250, 3255, 3260, 3265, 3270, 3275, 3280, 3285, 3290, 3295, 3300, 3305, 3310, 3315, 3320, 3325, 3330, 3335, 3340, 3345, 3350, 3355, 3360, 3365, 3370, 3375, 3380, 3385, 3390, 3395, 3400, 3405, 3410, 3415, 3420, 3425, 3430, 3435, 3440, 3445, 3450, 3455, 3460, 3465, 3470, 3475, 3480, 3485, 3490, 3495, 3500, 3505, 3510, 3515, 3520, 3525, 3530, 3535, 3540, 3545, 3550, 3555, 3560, 3565, 3570, 3575, 3580, 3585, 3590, 3595, 3600, 3605, 3610, 3615, 3620, 3625, 3630, 3635, 3640, 3645, 3650, 3655, 3660, 3665, 3670, 3675, 3680, 3685, 3690, 3695, 3700, 3705, 3710, 3715, 3720, 3725, 3730, 3735, 3740, 3745, 3750, 3755, 3760, 3765, 3770, 3775, 3780, 3785, 3790, 3795, 3800, 3805, 3810, 3815, 3820, 3825, 3830, 3835, 3840, 3845, 3850, 3855, 3860, 3865, 3870, 3875, 3880, 3885, 3890, 3895, 3900, 3905, 3910, 3915, 3920, 3925, 3930, 3935, 3940, 3945, 3950, 3955, 3960, 3965, 3970, 3975, 3980, 3985, 3990, 3995, 4000, 4010, 4015, 4020, 4025, 4030, 4035, 4040, 4045, 4050, 4055, 4060, 4065, 4070, 4075, 4080, 4085, 4090, 4095, 4100, 4105, 4110, 4115, 4120, 4125, 4130, 4135, 4140, 4145, 4150, 4155, 4160, 4165, 4170, 4175, 4180, 4185, 4190, 4195, 4200, 4205, 4210, 4215, 4220, 4225, 4230, 4235, 4240, 4245, 4250, 4255, 4260, 4265, 4270, 4275, 4280, 4285, 4290, 4295, 4300, 4305, 4310, 4315, 4320, 4325, 4330, 4335, 4340, 4345, 4350, 4355, 4360, 4365, 4370, 4375, 4380, 4385, 4390, 4395, 4400, 4405, 4410, 4415, 4420, 4425, 4430, 4435, 4440, 4445, 4450, 4455, 4460, 4465, 4470, 4475, 4480, 4485, 4490, 4495, 4500, 4505, 4510, 4515, 4520, 4525, 4530, 4535, 4540, 4545, 4550, 4555, 4560, 4565, 4570, 4575, 4580, 4585, 4590, 4595, 4600, 4605, 4610, 4615, 4620, 4625, 4630, 4635, 4640, 4645, 4650, 4655, 4660, 4665, 4670, 4675, 4680, 4685, 4690, 4695, 4700, 4705, 4710, 4715, 4720, 4725, 4730, 4735, 4740, 4745, 4750, 4755, 4760, 4765, 4770, 4775, 4780, 4785, 4790, 4795, 4800, 4805, 4810, 4815, 4820, 4825, 4830, 4835, 4840, 4845, 4850, 4855, 4860, 4865, 4870, 4875, 4880, 4885, 4890, 4895, 4900, 4905, 4910, 4915, 4920, 4925, 4930, 4935, 4940, 4945, 4950, 4955, 4960, 4965, 4970, 4975, 4980, 4985, 4990, 4995, 5000, 5005, 5010, 5015, 5020, 5025, 5030, 5035, 5040, 5045, 5050, 5055, 5060, 5065, 5070, 5075, 5080, 5085, 5090, 5095, 5100, 5105, 5110, 5115, 5120, 5125, 5130, 5135, 5140, 5145, 5150, 5155, 5160, 5165, 5170, 5175, 5180, 5185, 5190, 5195, 5200, 5205, 5210, 5215, 5220, 5225, 5230, 5235, 5240, 5245, 5250, 5255, 5260, 5265, 5270, 5275, 5280, 5285, 5290, 5295, 5300, 5305, 5310, 5315, 5320, 5325, 5330, 5335, 5340, 5345, 5350, 5355, 5360, 5365, 5370, 5375, 5380, 5385, 5390, 5395, 5400, 5405, 5410, 5415, 5420, 5425, 5430, 5435, 5440, 5445, 5450, 5455, 5460, 5465, 5470, 5475, 5480, 5485, 5490, 5495, 5500, 5505, 5510, 5515, 5520, 5525, 5530, 5535, 5540, 5545, 5550, 5555, 5560, 5565, 5570, 5575, 5580, 5585, 5590, 5595, 5600, 5605, 5610, 5615, 5620, 5625, 5630, 5635, 5640, 5645, 5650, 5655, 5660, 5665, 5670, 5675, 5680, 5685, 5690, 5695, 5700, 5705, 5710, 5715, 5720, 5725, 5730, 5735, 5740, 5745, 5750, 5755, 5760, 5765, 5770, 5775, 5780, 5785, 5790, 5795, 5800, 5805, 5810, 5815, 5820, 5825, 5830, 5835, 5840, 5845, 5850, 5855, 5860, 5865, 5870, 5875, 5880, 5885, 5890, 5895, 5900, 5905, 5910, 5915, 5920, 5925, 5930, 5935, 5940, 5945, 5950, 5955, 5960, 5965, 5970, 5975, 5980, 5985, 5990, 5995 and 6000 mg.

Preferably, for a body weight of 10-200 kg, the total dosage level (7-25 mg/kg) of anti-thrombin aptamer is 70-5000 mg. Preferably, for a body weight of 10-50 kg, the total dosage level is 70-1250 mg; for a body weight of 50-100 kg, the total dosage level is 350-2500 mg; for a body weight of 100-150 kg, the total dosage level is 700-3750 mg; and for a body weight of 150-200 kg, the total dosage level is 1050-5000 mg. The total dose may be any one of 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995, 1000, 1005, 1010, 1015, 1020, 1025, 1030, 1035, 1040, 1045, 1050, 1055, 1060, 1065, 1070, 1075, 1080, 1085, 1090, 1095, 1100, 1105, 1110, 1115, 1120, 1125, 1130, 1135, 1140, 1145, 1150, 1155, 1160, 1165, 1170, 1175, 1180, 1185, 1190, 1195, 1200, 1205, 1210, 1215, 1220, 1225, 1230, 1235, 1240, 1245, 1250, 1255, 1260, 1265, 1270, 1275, 1280, 1285, 1290, 1295, 1300, 1305, 1310, 1315, 1320, 1325, 1330, 1335, 1340, 1345, 1350, 1355, 1360, 1365, 1370, 1375, 1380, 1385, 1390, 1395, 1400, 1405, 1410, 1415, 1420, 1425, 1430, 1435, 1440, 1445, 1450, 1455, 1460, 1465, 1470, 1475, 1480, 1485, 1490, 1495, 1500, 1505, 1510, 1515, 1520, 1525, 1530, 1535, 1540, 1545, 1550, 1555, 1560, 1565, 1570, 1575, 1580, 1585, 1590, 1595, 1600, 1605, 1610, 1615, 1620, 1625, 1630, 1635, 1640, 1645, 1650, 1655, 1660, 1665, 1670, 1675, 1680, 1685, 1690, 1695, 1700, 1705, 1710, 1715, 1720, 1725, 1730, 1735, 1740, 1745, 1750, 1755, 1760, 1765, 1770, 1775, 1780, 1785, 1790, 1795, 1800, 1805, 1810, 1815, 1820, 1825, 1830, 1835, 1840, 1845, 1850, 1855, 1860, 1865, 1870, 1875, 1880, 1885, 1890, 1895, 1900, 1905, 1910, 1915, 1920, 1925, 1930, 1935, 1940, 1945, 1950, 1955, 1960, 1965, 1970, 1975, 1980, 1985, 1990, 1995, 2000, 2010, 2015, 2020, 2025, 2030, 2035, 2040, 2045, 2050, 2055, 2060, 2065, 2070, 2075, 2080, 2085, 2090, 2095, 2100, 2105, 2110, 2115, 2120, 2125, 2130, 2135, 2140, 2145, 2150, 2155, 2160, 2165, 2170, 2175, 2180, 2185, 2190, 2195, 2200, 2205, 2210, 2215, 2220, 2225, 2230, 2235, 2240, 2245, 2250, 2255, 2260, 2265, 2270, 2275, 2280, 2285, 2290, 2295, 2300, 2305, 2310, 2315, 2320, 2325, 2330, 2335, 2340, 2345, 2350, 2355, 2360, 2365, 2370, 2375, 2380, 2385, 2390, 2395, 2400, 2405, 2410, 2415, 2420, 2425, 2430, 2435, 2440, 2445, 2450, 2455, 2460, 2465, 2470, 2475, 2480, 2485, 2490, 2495, 2500, 2505, 2510, 2515, 2520, 2525, 2530, 2535, 2540, 2545, 2550, 2555, 2560, 2565, 2570, 2575, 2580, 2585, 2590, 2595, 2600, 2605, 2610, 2615, 2620, 2625, 2630, 2635, 2640, 2645, 2650, 2655, 2660, 2665, 2670, 2675, 2680, 2685, 2690, 2695, 2700, 2705, 2710, 2715, 2720, 2725, 2730, 2735, 2740, 2745, 2750, 2755, 2760, 2765, 2770, 2775, 2780, 2785, 2790, 2795, 2800, 2805, 2810, 2815, 2820, 2825, 2830, 2835, 2840, 2845, 2850, 2855, 2860, 2865, 2870, 2875, 2880, 2885, 2890, 2895, 2900, 2905, 2910, 2915, 2920, 2925, 2930, 2935, 2940, 2945, 2950, 2955, 2960, 2965, 2970, 2975, 2980, 2985, 2990, 2995, 3000, 3010, 3015, 3020, 3025, 3030, 3035, 3040, 3045, 3050, 3055, 3060, 3065, 3070, 3075, 3080, 3085, 3090, 3095, 3100, 3105, 3110, 3115, 3120, 3125, 3130, 3135, 3140, 3145, 3150, 3155, 3160, 3165, 3170, 3175, 3180, 3185, 3190, 3195, 3200, 3205, 3210, 3215, 3220, 3225, 3230, 3235, 3240, 3245, 3250, 3255, 3260, 3265, 3270, 3275, 3280, 3285, 3290, 3295, 3300, 3305, 3310, 3315, 3320, 3325, 3330, 3335, 3340, 3345, 3350, 3355, 3360, 3365, 3370, 3375, 3380, 3385, 3390, 3395, 3400, 3405, 3410, 3415, 3420, 3425, 3430, 3435, 3440, 3445, 3450, 3455, 3460, 3465, 3470, 3475, 3480, 3485, 3490, 3495, 3500, 3505, 3510, 3515, 3520, 3525, 3530, 3535, 3540, 3545, 3550, 3555, 3560, 3565, 3570, 3575, 3580, 3585, 3590, 3595, 3600, 3605, 3610, 3615, 3620, 3625, 3630, 3635, 3640, 3645, 3650, 3655, 3660, 3665, 3670, 3675, 3680, 3685, 3690, 3695, 3700, 3705, 3710, 3715, 3720, 3725, 3730, 3735, 3740, 3745, 3750, 3755, 3760, 3765, 3770, 3775, 3780, 3785, 3790, 3795, 3800, 3805, 3810, 3815, 3820, 3825, 3830, 3835, 3840, 3845, 3850, 3855, 3860, 3865, 3870, 3875, 3880, 3885, 3890, 3895, 3900, 3905, 3910, 3915, 3920, 3925, 3930, 3935, 3940, 3945, 3950, 3955, 3960, 3965, 3970, 3975, 3980, 3985, 3990, 3995, 4000, 4010, 4015, 4020, 4025, 4030, 4035, 4040, 4045, 4050, 4055, 4060, 4065, 4070, 4075, 4080, 4085, 4090, 4095, 4100, 4105, 4110, 4115, 4120, 4125, 4130, 4135, 4140, 4145, 4150, 4155, 4160, 4165, 4170, 4175, 4180, 4185, 4190, 4195, 4200, 4205, 4210, 4215, 4220, 4225, 4230, 4235, 4240, 4245, 4250, 4255, 4260, 4265, 4270, 4275, 4280, 4285, 4290, 4295, 4300, 4305, 4310, 4315, 4320, 4325, 4330, 4335, 4340, 4345, 4350, 4355, 4360, 4365, 4370, 4375, 4380, 4385, 4390, 4395, 4400, 4405, 4410, 4415, 4420, 4425, 4430, 4435, 4440, 4445, 4450, 4455, 4460, 4465, 4470, 4475, 4480, 4485, 4490, 4495, 4500, 4505, 4510, 4515, 4520, 4525, 4530, 4535, 4540, 4545, 4550, 4555, 4560, 4565, 4570, 4575, 4580, 4585, 4590, 4595, 4600, 4605, 4610, 4615, 4620, 4625, 4630, 4635, 4640, 4645, 4650, 4655, 4660, 4665, 4670, 4675, 4680, 4685, 4690, 4695, 4700, 4705, 4710, 4715, 4720, 4725, 4730, 4735, 4740, 4745, 4750, 4755, 4760, 4765, 4770, 4775, 4780, 4785, 4790, 4795, 4800, 4805, 4810, 4815, 4820, 4825, 4830, 4835, 4840, 4845, 4850, 4855, 4860, 4865, 4870, 4875, 4880, 4885, 4890, 4895, 4900, 4905, 4910, 4915, 4920, 4925, 4930, 4935, 4940, 4945, 4950, 4955, 4960, 4965, 4970, 4975, 4980, 4985, 4990, 4995 and 5000 mg.

More preferably, for a body weight of 10-200 kg, the total dosage level (9-20 mg/kg) of anti-thrombin aptamer is 90-4000 mg. Preferably, for a body weight of 10-50 kg, the total dosage level is 90-1000 mg; for a body weight of 50-100 kg, the total dosage level is 450-2000 mg; for a body weight of 100-150, the total dosage level is 900-3000 mg; and for a body weight of 150-200 kg, the total dosage level is 1350-4000 mg. The total dose may be any one of 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995, 1000, 1005, 1010, 1015, 1020, 1025, 1030, 1035, 1040, 1045, 1050, 1055, 1060, 1065, 1070, 1075, 1080, 1085, 1090, 1095, 1100, 1105, 1110, 1115, 1120, 1125, 1130, 1135, 1140, 1145, 1150, 1155, 1160, 1165, 1170, 1175, 1180, 1185, 1190, 1195, 1200, 1205, 1210, 1215, 1220, 1225, 1230, 1235, 1240, 1245, 1250, 1255, 1260, 1265, 1270, 1275, 1280, 1285, 1290, 1295, 1300, 1305, 1310, 1315, 1320, 1325, 1330, 1335, 1340, 1345, 1350, 1355, 1360, 1365, 1370, 1375, 1380, 1385, 1390, 1395, 1400, 1405, 1410, 1415, 1420, 1425, 1430, 1435, 1440, 1445, 1450, 1455, 1460, 1465, 1470, 1475, 1480, 1485, 1490, 1495, 1500, 1505, 1510, 1515, 1520, 1525, 1530, 1535, 1540, 1545, 1550, 1555, 1560, 1565, 1570, 1575, 1580, 1585, 1590, 1595, 1600, 1605, 1610, 1615, 1620, 1625, 1630, 1635, 1640, 1645, 1650, 1655, 1660, 1665, 1670, 1675, 1680, 1685, 1690, 1695, 1700, 1705, 1710, 1715, 1720, 1725, 1730, 1735, 1740, 1745, 1750, 1755, 1760, 1765, 1770, 1775, 1780, 1785, 1790, 1795, 1800, 1805, 1810, 1815, 1820, 1825, 1830, 1835, 1840, 1845, 1850, 1855, 1860, 1865, 1870, 1875, 1880, 1885, 1890, 1895, 1900, 1905, 1910, 1915, 1920, 1925, 1930, 1935, 1940, 1945, 1950, 1955, 1960, 1965, 1970, 1975, 1980, 1985, 1990, 1995, 2000, 2010, 2015, 2020, 2025, 2030, 2035, 2040, 2045, 2050, 2055, 2060, 2065, 2070, 2075, 2080, 2085, 2090, 2095, 2100, 2105, 2110, 2115, 2120, 2125, 2130, 2135, 2140, 2145, 2150, 2155, 2160, 2165, 2170, 2175, 2180, 2185, 2190, 2195, 2200, 2205, 2210, 2215, 2220, 2225, 2230, 2235, 2240, 2245, 2250, 2255, 2260, 2265, 2270, 2275, 2280, 2285, 2290, 2295, 2300, 2305, 2310, 2315, 2320, 2325, 2330, 2335, 2340, 2345, 2350, 2355, 2360, 2365, 2370, 2375, 2380, 2385, 2390, 2395, 2400, 2405, 2410, 2415, 2420, 2425, 2430, 2435, 2440, 2445, 2450, 2455, 2460, 2465, 2470, 2475, 2480, 2485, 2490, 2495, 2500, 2505, 2510, 2515, 2520, 2525, 2530, 2535, 2540, 2545, 2550, 2555, 2560, 2565, 2570, 2575, 2580, 2585, 2590, 2595, 2600, 2605, 2610, 2615, 2620, 2625, 2630, 2635, 2640, 2645, 2650, 2655, 2660, 2665, 2670, 2675, 2680, 2685, 2690, 2695, 2700, 2705, 2710, 2715, 2720, 2725, 2730, 2735, 2740, 2745, 2750, 2755, 2760, 2765, 2770, 2775, 2780, 2785, 2790, 2795, 2800, 2805, 2810, 2815, 2820, 2825, 2830, 2835, 2840, 2845, 2850, 2855, 2860, 2865, 2870, 2875, 2880, 2885, 2890, 2895, 2900, 2905, 2910, 2915, 2920, 2925, 2930, 2935, 2940, 2945, 2950, 2955, 2960, 2965, 2970, 2975, 2980, 2985, 2990, 2995, 3000, 3010, 3015, 3020, 3025, 3030, 3035, 3040, 3045, 3050, 3055, 3060, 3065, 3070, 3075, 3080, 3085, 3090, 3095, 3100, 3105, 3110, 3115, 3120, 3125, 3130, 3135, 3140, 3145, 3150, 3155, 3160, 3165, 3170, 3175, 3180, 3185, 3190, 3195, 3200, 3205, 3210, 3215, 3220, 3225, 3230, 3235, 3240, 3245, 3250, 3255, 3260, 3265, 3270, 3275, 3280, 3285, 3290, 3295, 3300, 3305, 3310, 3315, 3320, 3325, 3330, 3335, 3340, 3345, 3350, 3355, 3360, 3365, 3370, 3375, 3380, 3385, 3390, 3395, 3400, 3405, 3410, 3415, 3420, 3425, 3430, 3435, 3440, 3445, 3450, 3455, 3460, 3465, 3470, 3475, 3480, 3485, 3490, 3495, 3500, 3505, 3510, 3515, 3520, 3525, 3530, 3535, 3540, 3545, 3550, 3555, 3560, 3565, 3570, 3575, 3580, 3585, 3590, 3595, 3600, 3605, 3610, 3615, 3620, 3625, 3630, 3635, 3640, 3645, 3650, 3655, 3660, 3665, 3670, 3675, 3680, 3685, 3690, 3695, 3700, 3705, 3710, 3715, 3720, 3725, 3730, 3735, 3740, 3745, 3750, 3755, 3760, 3765, 3770, 3775, 3780, 3785, 3790, 3795, 3800, 3805, 3810, 3815, 3820, 3825, 3830, 3835, 3840, 3845, 3850, 3855, 3860, 3865, 3870, 3875, 3880, 3885, 3890, 3895, 3900, 3905, 3910, 3915, 3920, 3925, 3930, 3935, 3940, 3945, 3950, 3955, 3960, 3965, 3970, 3975, 3980, 3985, 3990, 3995 and 4000 mg.

Even more preferably, for a body weight of 10-200 kg, the total dosage level (11-16 mg/kg) of anti-thrombin aptamer is 110-3200 mg. Preferably, for a body weight of 10-50 kg, the total dosage level is 110-800 mg; for a body weight of 50-100 kg, the total dosage level is 550-1600 mg; for a body weight of 100-150 kg, the total dosage level is 1100-2400 mg; and for a body weight of 150-200 kg, the total dosage level is 1650-3200 mg. The total dose may be any one of 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995, 1000, 1005, 1010, 1015, 1020, 1025, 1030, 1035, 1040, 1045, 1050, 1055, 1060, 1065, 1070, 1075, 1080, 1085, 1090, 1095, 1100, 1105, 1110, 1115, 1120, 1125, 1130, 1135, 1140, 1145, 1150, 1155, 1160, 1165, 1170, 1175, 1180, 1185, 1190, 1195, 1200, 1205, 1210, 1215, 1220, 1225, 1230, 1235, 1240, 1245, 1250, 1255, 1260, 1265, 1270, 1275, 1280, 1285, 1290, 1295, 1300, 1305, 1310, 1315, 1320, 1325, 1330, 1335, 1340, 1345, 1350, 1355, 1360, 1365, 1370, 1375, 1380, 1385, 1390, 1395, 1400, 1405, 1410, 1415, 1420, 1425, 1430, 1435, 1440, 1445, 1450, 1455, 1460, 1465, 1470, 1475, 1480, 1485, 1490, 1495, 1500, 1505, 1510, 1515, 1520, 1525, 1530, 1535, 1540, 1545, 1550, 1555, 1560, 1565, 1570, 1575, 1580, 1585, 1590, 1595, 1600, 1605, 1610, 1615, 1620, 1625, 1630, 1635, 1640, 1645, 1650, 1655, 1660, 1665, 1670, 1675, 1680, 1685, 1690, 1695, 1700, 1705, 1710, 1715, 1720, 1725, 1730, 1735, 1740, 1745, 1750, 1755, 1760, 1765, 1770, 1775, 1780, 1785, 1790, 1795, 1800, 1805, 1810, 1815, 1820, 1825, 1830, 1835, 1840, 1845, 1850, 1855, 1860, 1865, 1870, 1875, 1880, 1885, 1890, 1895, 1900, 1905, 1910, 1915, 1920, 1925, 1930, 1935, 1940, 1945, 1950, 1955, 1960, 1965, 1970, 1975, 1980, 1985, 1990, 1995, 2000, 2010, 2015, 2020, 2025, 2030, 2035, 2040, 2045, 2050, 2055, 2060, 2065, 2070, 2075, 2080, 2085, 2090, 2095, 2100, 2105, 2110, 2115, 2120, 2125, 2130, 2135, 2140, 2145, 2150, 2155, 2160, 2165, 2170, 2175, 2180, 2185, 2190, 2195, 2200, 2205, 2210, 2215, 2220, 2225, 2230, 2235, 2240, 2245, 2250, 2255, 2260, 2265, 2270, 2275, 2280, 2285, 2290, 2295, 2300, 2305, 2310, 2315, 2320, 2325, 2330, 2335, 2340, 2345, 2350, 2355, 2360, 2365, 2370, 2375, 2380, 2385, 2390, 2395, 2400, 2405, 2410, 2415, 2420, 2425, 2430, 2435, 2440, 2445, 2450, 2455, 2460, 2465, 2470, 2475, 2480, 2485, 2490, 2495, 2500, 2505, 2510, 2515, 2520, 2525, 2530, 2535, 2540, 2545, 2550, 2555, 2560, 2565, 2570, 2575, 2580, 2585, 2590, 2595, 2600, 2605, 2610, 2615, 2620, 2625, 2630, 2635, 2640, 2645, 2650, 2655, 2660, 2665, 2670, 2675, 2680, 2685, 2690, 2695, 2700, 2705, 2710, 2715, 2720, 2725, 2730, 2735, 2740, 2745, 2750, 2755, 2760, 2765, 2770, 2775, 2780, 2785, 2790, 2795, 2800, 2805, 2810, 2815, 2820, 2825, 2830, 2835, 2840, 2845, 2850, 2855, 2860, 2865, 2870, 2875, 2880, 2885, 2890, 2895, 2900, 2905, 2910, 2915, 2920, 2925, 2930, 2935, 2940, 2945, 2950, 2955, 2960, 2965, 2970, 2975, 2980, 2985, 2990, 2995, 3000, 3010, 3015, 3020, 3025, 3030, 3035, 3040, 3045, 3050, 3055, 3060, 3065, 3070, 3075, 3080, 3085, 3090, 3095, 3100, 3105, 3110, 3115, 3120, 3125, 3130, 3135, 3140, 3145, 3150, 3155, 3160, 3165, 3170, 3175, 3180, 3185, 3190, 3195 and 3200 mg.

Most preferably, for a body weight of 10-200 kg, the total dosage level (12-15 mg/kg) of anti-thrombin aptamer is 120-3000 mg. Preferably, for a body weight of 10-50 kg, the total dosage level is 120-750 mg; for a body weight of 50-100 kg, the total dosage level is 600-1500 mg; for a body weight of 100-150 kg, the total dosage level is 1200-2250 mg; and for a body weight of 150-200 kg, the total dosage level is 1800-3000 mg. The total dose may be any one of 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995, 1000, 1005, 1010, 1015, 1020, 1025, 1030, 1035, 1040, 1045, 1050, 1055, 1060, 1065, 1070, 1075, 1080, 1085, 1090, 1095, 1100, 1105, 1110, 1115, 1120, 1125, 1130, 1135, 1140, 1145, 1150, 1155, 1160, 1165, 1170, 1175, 1180, 1185, 1190, 1195, 1200, 1205, 1210, 1215, 1220, 1225, 1230, 1235, 1240, 1245, 1250, 1255, 1260, 1265, 1270, 1275, 1280, 1285, 1290, 1295, 1300, 1305, 1310, 1315, 1320, 1325, 1330, 1335, 1340, 1345, 1350, 1355, 1360, 1365, 1370, 1375, 1380, 1385, 1390, 1395, 1400, 1405, 1410, 1415, 1420, 1425, 1430, 1435, 1440, 1445, 1450, 1455, 1460, 1465, 1470, 1475, 1480, 1485, 1490, 1495, 1500, 1505, 1510, 1515, 1520, 1525, 1530, 1535, 1540, 1545, 1550, 1555, 1560, 1565, 1570, 1575, 1580, 1585, 1590, 1595, 1600, 1605, 1610, 1615, 1620, 1625, 1630, 1635, 1640, 1645, 1650, 1655, 1660, 1665, 1670, 1675, 1680, 1685, 1690, 1695, 1700, 1705, 1710, 1715, 1720, 1725, 1730, 1735, 1740, 1745, 1750, 1755, 1760, 1765, 1770, 1775, 1780, 1785, 1790, 1795, 1800, 1805, 1810, 1815, 1820, 1825, 1830, 1835, 1840, 1845, 1850, 1855, 1860, 1865, 1870, 1875, 1880, 1885, 1890, 1895, 1900, 1905, 1910, 1915, 1920, 1925, 1930, 1935, 1940, 1945, 1950, 1955, 1960, 1965, 1970, 1975, 1980, 1985, 1990, 1995, 2000, 2010, 2015, 2020, 2025, 2030, 2035, 2040, 2045, 2050, 2055, 2060, 2065, 2070, 2075, 2080, 2085, 2090, 2095, 2100, 2105, 2110, 2115, 2120, 2125, 2130, 2135, 2140, 2145, 2150, 2155, 2160, 2165, 2170, 2175, 2180, 2185, 2190, 2195, 2200, 2205, 2210, 2215, 2220, 2225, 2230, 2235, 2240, 2245, 2250, 2255, 2260, 2265, 2270, 2275, 2280, 2285, 2290, 2295, 2300, 2305, 2310, 2315, 2320, 2325, 2330, 2335, 2340, 2345, 2350, 2355, 2360, 2365, 2370, 2375, 2380, 2385, 2390, 2395, 2400, 2405, 2410, 2415, 2420, 2425, 2430, 2435, 2440, 2445, 2450, 2455, 2460, 2465, 2470, 2475, 2480, 2485, 2490, 2495, 2500, 2505, 2510, 2515, 2520, 2525, 2530, 2535, 2540, 2545, 2550, 2555, 2560, 2565, 2570, 2575, 2580, 2585, 2590, 2595, 2600, 2605, 2610, 2615, 2620, 2625, 2630, 2635, 2640, 2645, 2650, 2655, 2660, 2665, 2670, 2675, 2680, 2685, 2690, 2695, 2700, 2705, 2710, 2715, 2720, 2725, 2730, 2735, 2740, 2745, 2750, 2755, 2760, 2765, 2770, 2775, 2780, 2785, 2790, 2795, 2800, 2805, 2810, 2815, 2820, 2825, 2830, 2835, 2840, 2845, 2850, 2855, 2860, 2865, 2870, 2875, 2880, 2885, 2890, 2895, 2900, 2905, 2910, 2915, 2920, 2925, 2930, 2935, 2940, 2945, 2950, 2955, 2960, 2965, 2970, 2975, 2980, 2985, 2990, 2995 and 3000 mg.

### Administration

ARC2172 is manufactured for clinical use as a sterile isotonic saline solution (0.9% saline solution) for injection. Preferably, the formulation is provided in a 15 mg/mL solution, at pH 7.0±0.4. The formulation may be administered directly into an individual or may be diluted into an IV bag prior to administration.

The formulations are suitable for parenteral administration. Suitable routes for parenteral administration include intravenous, subcutaneous, intradermal, intramuscular, intraarticular and intrathecal administration. Suitable routes of administration may also be used in combination, such as intravenous administration followed by subcutaneous administration. The route of administration, however, is determined by the attending physician. Preferably, the formulations are administered intravenously.

The formulations may be administered parenterally, for example, as a bolus; a slow bolus over a short period of time, such as 15 minutes; a continual infusion or a continual drip. Preferably, the formulations are administered by continual infusion.

Administration by continual infusion may be at a constant rate. Alternatively, the rate of administration may be varied (not constant) over time in order to take into account a loading dose or doses prior to or at the beginning of administration and tapering of the infusion rate at the end of administration. Preferably, the rate of continual infusion is varied.

### Preferred Dosing and Administration

As stated above, preferably, a total dose of anti-thrombin aptamer of 7-25 mg/kg is administered. More preferably, a total dose of 9-20 mg/kg is administered. Even more preferably, a total dose of 11-16 mg/kg is administered. Most preferably, a total dose of 12-15 mg/kg is administered. This total dosage may be administered in a single dose, multiple doses, as a continual dose or a combination thereof. For example, a loading dose or doses may be administered followed by a maintenance dose or doses. To achieve the desired blood concentration level, any total dose of 1.0-30.0 mg/kg can be used.

Preferably, the formulations are administered with a loading dose and a maintenance dose.

The loading dose of anti-thrombin aptamer is administered in mg per kg (mg/kg) of body weight. The loading dose of anti-thrombin aptamer is 0.2-2.5 mg/kg. The loading dose can be any dose between, and including, 0.2 mg/kg and 2.5 mg/kg in increments of 0.1 mg/kg. For example, acceptable loading doses include, but are not limited to, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1.0 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg, 2.1 mg/kg, 2.2 mg/kg, 2.3 mg/kg, 2.4 mg/kg and 2.5 mg/kg.

Preferably, the loading dose is a bolus intravenous infusion of anti-thrombin aptamer. Preferably, the loading dose is 0.2-2.5 mg/kg. More preferably, the loading dose is 0.75-2.25 mg/kg. Even more preferably, the loading dose is 1.0-2.0 mg/kg. Most preferably, the loading dose is 1.25-1.75 mg/kg. However, the dose and duration may be varied to achieve essentially the same result of a safe and tolerable dose that rapidly achieves the desired steady state concentration.

Preferably, for a body weight of 10-200 kg, the loading dose level (0.2-2.5 mg/kg) of anti-thrombin aptamer is 2-500 mg. Preferably, for a body weight of 10-50 kg, the loading dose level is 2-125 mg; for a body weight of 50-100 kg, the loading dose level is 10-250 mg; for a body weight of 100-150 kg, the loading dose level is 20-375 mg; and for a body weight of 150-200 kg, the loading dose level is 30-500 mg. The loading dose may be any one of 2, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495 and 500 mg.

More preferably, for a body weight of 10-200 kg, the loading dose level (0.75-2.25 mg/kg) of anti-thrombin aptamer is 7.5-450 mg. Preferably, for a body weight of 10-50 kg, the loading dose level is 7.5-112.5 mg; for a body weight of 50-100 kg, the loading dose level is 37.5-225 mg; for a body weight of 100-150 kg, the loading dose level is 75-337.5 mg; and for a body weight of 150-200 kg, the loading dose level is 112.5-450 mg. The loading dose may be any one of 7.5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445 and 450 mg.

Even more preferably, for a body weight of 10-200 kg, the loading dose level (1.0-2.0 mg/kg) of anti-thrombin aptamer is 10-400 mg. Preferably, for a body weight of 10-50 kg, the loading dose level is 10-100 mg; for a body weight of 50-100 kg, the loading dose level is 50-200 mg; for a body weight of 100-150 kg, the loading dose level is 100-300 mg; and for a body weight of 150-200 kg, the loading dose level is 150-400 mg. The loading dose may be any one of 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395 and 400 mg.

Most preferably, for a body weight of 10-200 kg, the loading dose level (1.25-1.75 mg/kg) of anti-thrombin aptamer is 12.5-350 mg. Preferably, for a body weight of 10-50 kg, the loading dose level is 12.5-87.5 mg; for a body weight of 50-100 kg, the loading dose level is 62.5-175 mg; for a body weight of 100-150 kg, the loading dose level is 125-262.5 mg; and for a body weight of 150-200 kg, the loading dose level is 187.5-350 mg. The loading dose may be any one of 12.5, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345 and 350 mg.

The maintenance dose of anti-thrombin aptamer is administered in mg per kg (mg/kg) of body weight. The maintenance dose of anti-thrombin aptamer is 1.0-30.0 mg/kg. The maintenance dose can be any dose between, and including, 1.0 mg/kg and 30.0 mg/kg in increments of 0.1 mg/kg. For example, acceptable maintenance dosage ranges include, but are not limited to, 1.0-2.0 mg/kg, 1.5-2.5 mg/kg, 2-3 mg/kg, 2.5-3.5 mg/kg, 3-4 mg/kg, 3.5-4.5 mg/kg, 4-5 mg/kg, 4.5-5.5 mg/kg, 5-6 mg/kg, 5.5-6.5 mg/kg, 6-7 mg/kg, 6.5-7.5 mg/kg, 7-8 mg/kg, 7.5-8.5 mg/kg, 8-9 mg/kg, 8.5-9.5 mg/kg, 9-10 mg/kg, 9.5-10.5 mg/kg, 10-11 mg/kg, 10.5-11.5 mg/kg, 11.0-12.0 mg/kg, 11.5-12.5 mg/kg, 12-13 mg/kg, 12.5-13.5 mg/kg, 13-14 mg/kg, 13.5-14.5 mg/kg, 14-15 mg/kg, 14.5-15.5 mg/kg, 15-16 mg/kg, 15.5-16.5 mg/kg, 16-17 mg/kg, 16.5-17.5 mg/kg, 17-18 mg/kg, 17.5-18.5 mg/kg, 18-19 mg/kg, 18.5-19.5 mg/kg, 19-20 mg/kg; 19.5-20.5 mg/kg, 20-21 mg/kg, 20.5-21.5 mg/kg, 21-22 mg/kg, 21.5-22.5 mg/kg, 22-23 mg/kg, 22.5-23.5 mg/kg, 23-24 mg/kg, 23.5-24.5 mg/kg, 24-25 mg/kg, 24.5-25.5 mg/kg, 25-26 mg/kg, 25.5-26.5 mg/kg, 26-27 mg/kg, 26.5-27.5 mg/kg, 27-28 mg/kg, 27.5-28.5 mg/kg, 28-29 mg/kg, 28.5-29.5 mg/kg, 29-30 mg/kg, and 29.5-30 mg/kg.

Preferably, the maintenance dose of anti-thrombin aptamer is administered as a continual infusion at a constant rate for four hours. Preferably, the maintenance dose is 6.0-25.0 mg/kg. More preferably, the maintenance dose is 8.0-18.0 mg/kg. Even more preferably, the maintenance dose is 10.0-16.0 mg/kg. Most preferably, the maintenance dose is 12.0-14.0 mg/kg. Preferably, the maintenance dose is administered as a continuous infusion until anticoagulation is no longer needed. Preferably, the maintenance dose is infused at a rate between 0.0281-0.1000 mg/kg/min. More preferably, the maintenance dose is infused at a rate between 0.0281-0.0771 mg/kg/min.

For example, for a body weight of 10-200 kg, the maintenance dosage level (1-30 mg/kg) of anti-thrombin aptamer is 10-6000 mg. For an average body weight of 70 kg, the maintenance dosage level is 70-2100 mg. The maintenance dose may be any one of 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995, 1000, 1005, 1010, 1015, 1020, 1025, 1030, 1035, 1040, 1045, 1050, 1055, 1060, 1065, 1070, 1075, 1080, 1085, 1090, 1095, 1100, 1105, 1110, 1115, 1120, 1125, 1130, 1135, 1140, 1145, 1150, 1155, 1160, 1165, 1170, 1175, 1180, 1185, 1190, 1195, 1200, 1205, 1210, 1215, 1220, 1225, 1230, 1235, 1240, 1245, 1250, 1255, 1260, 1265, 1270, 1275, 1280, 1285, 1290, 1295, 1300, 1305, 1310, 1315, 1320, 1325, 1330, 1335, 1340, 1345, 1350, 1355, 1360, 1365, 1370, 1375, 1380, 1385, 1390, 1395, 1400, 1405, 1410, 1415, 1420, 1425, 1430, 1435, 1440, 1445, 1450, 1455, 1460, 1465, 1470, 1475, 1480, 1485, 1490, 1495, 1500, 1505, 1510, 1515, 1520, 1525, 1530, 1535, 1540, 1545, 1550, 1555, 1560, 1565, 1570, 1575, 1580, 1585, 1590, 1595, 1600, 1605, 1610, 1615, 1620, 1625, 1630, 1635, 1640, 1645, 1650, 1655, 1660, 1665, 1670, 1675, 1680, 1685, 1690, 1695, 1700, 1705, 1710, 1715, 1720, 1725, 1730, 1735, 1740, 1745, 1750, 1755, 1760, 1765, 1770, 1775, 1780, 1785, 1790, 1795, 1800, 1805, 1810, 1815, 1820, 1825, 1830, 1835, 1840, 1845, 1850, 1855, 1860, 1865, 1870, 1875, 1880, 1885, 1890, 1895, 1900, 1905, 1910, 1915, 1920, 1925, 1930, 1935, 1940, 1945, 1950, 1955, 1960, 1965, 1970, 1975, 1980, 1985, 1990, 1995, 2000, 2010, 2015, 2020, 2025, 2030, 2035, 2040, 2045, 2050, 2055, 2060, 2065, 2070, 2075, 2080, 2085, 2090, 2095, 2100, 2105, 2110, 2115, 2120, 2125, 2130, 2135, 2140, 2145, 2150, 2155, 2160, 2165, 2170, 2175, 2180, 2185, 2190, 2195, 2200, 2205, 2210, 2215, 2220, 2225, 2230, 2235, 2240, 2245, 2250, 2255, 2260, 2265, 2270, 2275, 2280, 2285, 2290, 2295, 2300, 2305, 2310, 2315, 2320, 2325, 2330, 2335, 2340, 2345, 2350, 2355, 2360, 2365, 2370, 2375, 2380, 2385, 2390, 2395, 2400, 2405, 2410, 2415, 2420, 2425, 2430, 2435, 2440, 2445, 2450, 2455, 2460, 2465, 2470, 2475, 2480, 2485, 2490, 2495, 2500, 2505, 2510, 2515, 2520, 2525, 2530, 2535, 2540, 2545, 2550, 2555, 2560, 2565, 2570, 2575, 2580, 2585, 2590, 2595, 2600, 2605, 2610, 2615, 2620, 2625, 2630, 2635, 2640, 2645, 2650, 2655, 2660, 2665, 2670, 2675, 2680, 2685, 2690, 2695, 2700, 2705, 2710, 2715, 2720, 2725, 2730, 2735, 2740, 2745, 2750, 2755, 2760, 2765, 2770, 2775, 2780, 2785, 2790, 2795, 2800, 2805, 2810, 2815, 2820, 2825, 2830, 2835, 2840, 2845, 2850, 2855, 2860, 2865, 2870, 2875, 2880, 2885, 2890, 2895, 2900, 2905, 2910, 2915, 2920, 2925, 2930, 2935, 2940, 2945, 2950, 2955, 2960, 2965, 2970, 2975, 2980, 2985, 2990, 2995, 3000, 3010, 3015, 3020, 3025, 3030, 3035, 3040, 3045, 3050, 3055, 3060, 3065, 3070, 3075, 3080, 3085, 3090, 3095, 3100, 3105, 3110, 3115, 3120, 3125, 3130, 3135, 3140, 3145, 3150, 3155, 3160, 3165, 3170, 3175, 3180, 3185, 3190, 3195, 3200, 3205, 3210, 3215, 3220, 3225, 3230, 3235, 3240, 3245, 3250, 3255, 3260, 3265, 3270, 3275, 3280, 3285, 3290, 3295, 3300, 3305, 3310, 3315, 3320, 3325, 3330, 3335, 3340, 3345, 3350, 3355, 3360, 3365, 3370, 3375, 3380, 3385, 3390, 3395, 3400, 3405, 3410, 3415, 3420, 3425, 3430, 3435, 3440, 3445, 3450, 3455, 3460, 3465, 3470, 3475, 3480, 3485, 3490, 3495, 3500, 3505, 3510, 3515, 3520, 3525, 3530, 3535, 3540, 3545, 3550, 3555, 3560, 3565, 3570, 3575, 3580, 3585, 3590, 3595, 3600, 3605, 3610, 3615, 3620, 3625, 3630, 3635, 3640, 3645, 3650, 3655, 3660, 3665, 3670, 3675, 3680, 3685, 3690, 3695, 3700, 3705, 3710, 3715, 3720, 3725, 3730, 3735, 3740, 3745, 3750, 3755, 3760, 3765, 3770, 3775, 3780, 3785, 3790, 3795, 3800, 3805, 3810, 3815, 3820, 3825, 3830, 3835, 3840, 3845, 3850, 3855, 3860, 3865, 3870, 3875, 3880, 3885, 3890, 3895, 3900, 3905, 3910, 3915, 3920, 3925, 3930, 3935, 3940, 3945, 3950, 3955, 3960, 3965, 3970, 3975, 3980, 3985, 3990, 3995, 4000, 4010, 4015, 4020, 4025, 4030, 4035, 4040, 4045, 4050, 4055, 4060, 4065, 4070, 4075, 4080, 4085, 4090, 4095, 4100, 4105, 4110, 4115, 4120, 4125, 4130, 4135, 4140, 4145, 4150, 4155, 4160, 4165, 4170, 4175, 4180, 4185, 4190, 4195, 4200, 4205, 4210, 4215, 4220, 4225, 4230, 4235, 4240, 4245, 4250, 4255, 4260, 4265, 4270, 4275, 4280, 4285, 4290, 4295, 4300, 4305, 4310, 4315, 4320, 4325, 4330, 4335, 4340, 4345, 4350, 4355, 4360, 4365, 4370, 4375, 4380, 4385, 4390, 4395, 4400, 4405, 4410, 4415, 4420, 4425, 4430, 4435, 4440, 4445, 4450, 4455, 4460, 4465, 4470, 4475, 4480, 4485, 4490, 4495, 4500, 4505, 4510, 4515, 4520, 4525, 4530, 4535, 4540, 4545, 4550, 4555, 4560, 4565, 4570, 4575, 4580, 4585, 4590, 4595, 4600, 4605, 4610, 4615, 4620, 4625, 4630, 4635, 4640, 4645, 4650, 4655, 4660, 4665, 4670, 4675, 4680, 4685, 4690, 4695, 4700, 4705, 4710, 4715, 4720, 4725, 4730, 4735, 4740, 4745, 4750, 4755, 4760, 4765, 4770, 4775, 4780, 4785, 4790, 4795, 4800, 4805, 4810, 4815, 4820, 4825, 4830, 4835, 4840, 4845, 4850, 4855, 4860, 4865, 4870, 4875, 4880, 4885, 4890, 4895, 4900, 4905, 4910, 4915, 4920, 4925, 4930, 4935, 4940, 4945, 4950, 4955, 4960, 4965, 4970, 4975, 4980, 4985, 4990, 4995, 5000, 5005, 5010, 5015, 5020, 5025, 5030, 5035, 5040, 5045, 5050, 5055, 5060, 5065, 5070, 5075, 5080, 5085, 5090, 5095, 5100, 5105, 5110, 5115, 5120, 5125, 5130, 5135, 5140, 5145, 5150, 5155, 5160, 5165, 5170, 5175, 5180, 5185, 5190, 5195, 5200, 5205, 5210, 5215, 5220, 5225, 5230, 5235, 5240, 5245, 5250, 5255, 5260, 5265, 5270, 5275, 5280, 5285, 5290, 5295, 5300, 5305, 5310, 5315, 5320, 5325, 5330, 5335, 5340, 5345, 5350, 5355, 5360, 5365, 5370, 5375, 5380, 5385, 5390, 5395, 5400, 5405, 5410, 5415, 5420, 5425, 5430, 5435, 5440, 5445, 5450, 5455, 5460, 5465, 5470, 5475, 5480, 5485, 5490, 5495, 5500, 5505, 5510, 5515, 5520, 5525, 5530, 5535, 5540, 5545, 5550, 5555, 5560, 5565, 5570, 5575, 5580, 5585, 5590, 5595, 5600, 5605, 5610, 5615, 5620, 5625, 5630, 5635, 5640, 5645, 5650, 5655, 5660, 5665, 5670, 5675, 5680, 5685, 5690, 5695, 5700, 5705, 5710, 5715, 5720, 5725, 5730, 5735, 5740, 5745, 5750, 5755, 5760, 5765, 5770, 5775, 5780, 5785, 5790, 5795, 5800, 5805, 5810, 5815, 5820, 5825, 5830, 5835, 5840, 5845, 5850, 5855, 5860, 5865, 5870, 5875, 5880, 5885, 5890, 5895, 5900, 5905, 5910, 5915, 5920, 5925, 5930, 5935, 5940, 5945, 5950, 5955, 5960, 5965, 5970, 5975, 5980, 5985, 5990, 5995 and 6000 mg.

For example, for a body weight of 10-50 kg, the maintenance dosage level (1-30 mg/kg) of anti-thrombin aptamer is 10-1500 mg. The maintenance dose may be any one of 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995, 1000, 1005, 1010, 1015, 1020, 1025, 1030, 1035, 1040, 1045, 1050, 1055, 1060, 1065, 1070, 1075, 1080, 1085, 1090, 1095, 1100, 1105, 1110, 1115, 1120, 1125, 1130, 1135, 1140, 1145, 1150, 1155, 1160, 1165, 1170, 1175, 1180, 1185, 1190, 1195, 1200, 1205, 1210, 1215, 1220, 1225, 1230, 1235, 1240, 1245, 1250, 1255, 1260, 1265, 1270, 1275, 1280, 1285, 1290, 1295, 1300, 1305, 1310, 1315, 1320, 1325, 1330, 1335, 1340, 1345, 1350, 1355, 1360, 1365, 1370, 1375, 1380, 1385, 1390, 1395, 1400, 1405, 1410, 1415, 1420, 1425, 1430, 1435, 1440, 1445, 1450, 1455, 1460, 1465, 1470, 1475, 1480, 1485, 1490, 1495 and 1500 mg.

For example, for a body weight of 50-100 kg, the maintenance dosage level (1-30 mg/kg) of anti-thrombin aptamer is 50-3000 mg. The maintenance dose may be any one of 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995, 1000, 1005, 1010, 1015, 1020, 1025, 1030, 1035, 1040, 1045, 1050, 1055, 1060, 1065, 1070, 1075, 1080, 1085, 1090, 1095, 1100, 1105, 1110, 1115, 1120, 1125, 1130, 1135, 1140, 1145, 1150, 1155, 1160, 1165, 1170, 1175, 1180, 1185, 1190, 1195, 1200, 1205, 1210, 1215, 1220, 1225, 1230, 1235, 1240, 1245, 1250, 1255, 1260, 1265, 1270, 1275, 1280, 1285, 1290, 1295, 1300, 1305, 1310, 1315, 1320, 1325, 1330, 1335, 1340, 1345, 1350, 1355, 1360, 1365, 1370, 1375, 1380, 1385, 1390, 1395, 1400, 1405, 1410, 1415, 1420, 1425, 1430, 1435, 1440, 1445, 1450, 1455, 1460, 1465, 1470, 1475, 1480, 1485, 1490, 1495, 1500, 1505, 1510, 1515, 1520, 1525, 1530, 1535, 1540, 1545, 1550, 1555, 1560, 1565, 1570, 1575, 1580, 1585, 1590, 1595, 1600, 1605, 1610, 1615, 1620, 1625, 1630, 1635, 1640, 1645, 1650, 1655, 1660, 1665, 1670, 1675, 1680, 1685, 1690, 1695, 1700, 1705, 1710, 1715, 1720, 1725, 1730, 1735, 1740, 1745, 1750, 1755, 1760, 1765, 1770, 1775, 1780, 1785, 1790, 1795, 1800, 1805, 1810, 1815, 1820, 1825, 1830, 1835, 1840, 1845, 1850, 1855, 1860, 1865, 1870, 1875, 1880, 1885, 1890, 1895, 1900, 1905, 1910, 1915, 1920, 1925, 1930, 1935, 1940, 1945, 1950, 1955, 1960, 1965, 1970, 1975, 1980, 1985, 1990, 1995, 2000, 2010, 2015, 2020, 2025, 2030, 2035, 2040, 2045, 2050, 2055, 2060, 2065, 2070, 2075, 2080, 2085, 2090, 2095, 2100, 2105, 2110, 2115, 2120, 2125, 2130, 2135, 2140, 2145, 2150, 2155, 2160, 2165, 2170, 2175, 2180, 2185, 2190, 2195, 2200, 2205, 2210, 2215, 2220, 2225, 2230, 2235, 2240, 2245, 2250, 2255, 2260, 2265, 2270, 2275, 2280, 2285, 2290, 2295, 2300, 2305, 2310, 2315, 2320, 2325, 2330, 2335, 2340, 2345, 2350, 2355, 2360, 2365, 2370, 2375, 2380, 2385, 2390, 2395, 2400, 2405, 2410, 2415, 2420, 2425, 2430, 2435, 2440, 2445, 2450, 2455, 2460, 2465, 2470, 2475, 2480, 2485, 2490, 2495, 2500, 2505, 2510, 2515, 2520, 2525, 2530, 2535, 2540, 2545, 2550, 2555, 2560, 2565, 2570, 2575, 2580, 2585, 2590, 2595, 2600, 2605, 2610, 2615, 2620, 2625, 2630, 2635, 2640, 2645, 2650, 2655, 2660, 2665, 2670, 2675, 2680, 2685, 2690, 2695, 2700, 2705, 2710, 2715, 2720, 2725, 2730, 2735, 2740, 2745, 2750, 2755, 2760, 2765, 2770, 2775, 2780, 2785, 2790, 2795, 2800, 2805, 2810, 2815, 2820, 2825, 2830, 2835, 2840, 2845, 2850, 2855, 2860, 2865, 2870, 2875, 2880, 2885, 2890, 2895, 2900, 2905, 2910, 2915, 2920, 2925, 2930, 2935, 2940, 2945, 2950, 2955, 2960, 2965, 2970, 2975, 2980, 2985, 2990, 2995 and 3000 mg.

For example, for a body weight of 100-150 kg, the maintenance dosage level (1-30 mg/kg) of anti-thrombin aptamer is 100-4500 mg. The maintenance dose may be any one of 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995, 1000, 1005, 1010, 1015, 1020, 1025, 1030, 1035, 1040, 1045, 1050, 1055, 1060, 1065, 1070, 1075, 1080, 1085, 1090, 1095, 1100, 1105, 1110, 1115, 1120, 1125, 1130, 1135, 1140, 1145, 1150, 1155, 1160, 1165, 1170, 1175, 1180, 1185, 1190, 1195, 1200, 1205, 1210, 1215, 1220, 1225, 1230, 1235, 1240, 1245, 1250, 1255, 1260, 1265, 1270, 1275, 1280, 1285, 1290, 1295, 1300, 1305, 1310, 1315, 1320, 1325, 1330, 1335, 1340, 1345, 1350, 1355, 1360, 1365, 1370, 1375, 1380, 1385, 1390, 1395, 1400, 1405, 1410, 1415, 1420, 1425, 1430, 1435, 1440, 1445, 1450, 1455, 1460, 1465, 1470, 1475, 1480, 1485, 1490, 1495, 1500, 1505, 1510, 1515, 1520, 1525, 1530, 1535, 1540, 1545, 1550, 1555, 1560, 1565, 1570, 1575, 1580, 1585, 1590, 1595, 1600, 1605, 1610, 1615, 1620, 1625, 1630, 1635, 1640, 1645, 1650, 1655, 1660, 1665, 1670, 1675, 1680, 1685, 1690, 1695, 1700, 1705, 1710, 1715, 1720, 1725, 1730, 1735, 1740, 1745, 1750, 1755, 1760, 1765, 1770, 1775, 1780, 1785, 1790, 1795, 1800, 1805, 1810, 1815, 1820, 1825, 1830, 1835, 1840, 1845, 1850, 1855, 1860, 1865, 1870, 1875, 1880, 1885, 1890, 1895, 1900, 1905, 1910, 1915, 1920, 1925, 1930, 1935, 1940, 1945, 1950, 1955, 1960, 1965, 1970, 1975, 1980, 1985, 1990, 1995, 2000, 2010, 2015, 2020, 2025, 2030, 2035, 2040, 2045, 2050, 2055, 2060, 2065, 2070, 2075, 2080, 2085, 2090, 2095, 2100, 2105, 2110, 2115, 2120, 2125, 2130, 2135, 2140, 2145, 2150, 2155, 2160, 2165, 2170, 2175, 2180, 2185, 2190, 2195, 2200, 2205, 2210, 2215, 2220, 2225, 2230, 2235, 2240, 2245, 2250, 2255, 2260, 2265, 2270, 2275, 2280, 2285, 2290, 2295, 2300, 2305, 2310, 2315, 2320, 2325, 2330, 2335, 2340, 2345, 2350, 2355, 2360, 2365, 2370, 2375, 2380, 2385, 2390, 2395, 2400, 2405, 2410, 2415, 2420, 2425, 2430, 2435, 2440, 2445, 2450, 2455, 2460, 2465, 2470, 2475, 2480, 2485, 2490, 2495, 2500, 2505, 2510, 2515, 2520, 2525, 2530, 2535, 2540, 2545, 2550, 2555, 2560, 2565, 2570, 2575, 2580, 2585, 2590, 2595, 2600, 2605, 2610, 2615, 2620, 2625, 2630, 2635, 2640, 2645, 2650, 2655, 2660, 2665, 2670, 2675, 2680, 2685, 2690, 2695, 2700, 2705, 2710, 2715, 2720, 2725, 2730, 2735, 2740, 2745, 2750, 2755, 2760, 2765, 2770, 2775, 2780, 2785, 2790, 2795, 2800, 2805, 2810, 2815, 2820, 2825, 2830, 2835, 2840, 2845, 2850, 2855, 2860, 2865, 2870, 2875, 2880, 2885, 2890, 2895, 2900, 2905, 2910, 2915, 2920, 2925, 2930, 2935, 2940, 2945, 2950, 2955, 2960, 2965, 2970, 2975, 2980, 2985, 2990, 2995, 3000, 3010, 3015, 3020, 3025, 3030, 3035, 3040, 3045, 3050, 3055, 3060, 3065, 3070, 3075, 3080, 3085, 3090, 3095, 3100, 3105, 3110, 3115, 3120, 3125, 3130, 3135, 3140, 3145, 3150, 3155, 3160, 3165, 3170, 3175, 3180, 3185, 3190, 3195, 3200, 3205, 3210, 3215, 3220, 3225, 3230, 3235, 3240, 3245, 3250, 3255, 3260, 3265, 3270, 3275, 3280, 3285, 3290, 3295, 3300, 3305, 3310, 3315, 3320, 3325, 3330, 3335, 3340, 3345, 3350, 3355, 3360, 3365, 3370, 3375, 3380, 3385, 3390, 3395, 3400, 3405, 3410, 3415, 3420, 3425, 3430, 3435, 3440, 3445, 3450, 3455, 3460, 3465, 3470, 3475, 3480, 3485, 3490, 3495, 3500, 3505, 3510, 3515, 3520, 3525, 3530, 3535, 3540, 3545, 3550, 3555, 3560, 3565, 3570, 3575, 3580, 3585, 3590, 3595, 3600, 3605, 3610, 3615, 3620, 3625, 3630, 3635, 3640, 3645, 3650, 3655, 3660, 3665, 3670, 3675, 3680, 3685, 3690, 3695, 3700, 3705, 3710, 3715, 3720, 3725, 3730, 3735, 3740, 3745, 3750, 3755, 3760, 3765, 3770, 3775, 3780, 3785, 3790, 3795, 3800, 3805, 3810, 3815, 3820, 3825, 3830, 3835, 3840, 3845, 3850, 3855, 3860, 3865, 3870, 3875, 3880, 3885, 3890, 3895, 3900, 3905, 3910, 3915, 3920, 3925, 3930, 3935, 3940, 3945, 3950, 3955, 3960, 3965, 3970, 3975, 3980, 3985, 3990, 3995, 4000, 4010, 4015, 4020, 4025, 4030, 4035, 4040, 4045, 4050, 4055, 4060, 4065, 4070, 4075, 4080, 4085, 4090, 4095, 4100, 4105, 4110, 4115, 4120, 4125, 4130, 4135, 4140, 4145, 4150, 4155, 4160, 4165, 4170, 4175, 4180, 4185, 4190, 4195, 4200, 4205, 4210, 4215, 4220, 4225, 4230, 4235, 4240, 4245, 4250, 4255, 4260, 4265, 4270, 4275, 4280, 4285, 4290, 4295, 4300, 4305, 4310, 4315, 4320, 4325, 4330, 4335, 4340, 4345, 4350, 4355, 4360, 4365, 4370, 4375, 4380, 4385, 4390, 4395, 4400, 4405, 4410, 4415, 4420, 4425, 4430, 4435, 4440, 4445, 4450, 4455, 4460, 4465, 4470, 4475, 4480, 4485, 4490, 4495 and 4500 mg.

For example, for a body weight of 150-200 kg, the maintenance dosage level (1-30 mg/kg) of anti-thrombin aptamer is 150-6000 mg. The maintenance dose may be any one of 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995, 1000, 1005, 1010, 1015, 1020, 1025, 1030, 1035, 1040, 1045, 1050, 1055, 1060, 1065, 1070, 1075, 1080, 1085, 1090, 1095, 1100, 1105, 1110, 1115, 1120, 1125, 1130, 1135, 1140, 1145, 1150, 1155, 1160, 1165, 1170, 1175, 1180, 1185, 1190, 1195, 1200, 1205, 1210, 1215, 1220, 1225, 1230, 1235, 1240, 1245, 1250, 1255, 1260, 1265, 1270, 1275, 1280, 1285, 1290, 1295, 1300, 1305, 1310, 1315, 1320, 1325, 1330, 1335, 1340, 1345, 1350, 1355, 1360, 1365, 1370, 1375, 1380, 1385, 1390, 1395, 1400, 1405, 1410, 1415, 1420, 1425, 1430, 1435, 1440, 1445, 1450, 1455, 1460, 1465, 1470, 1475, 1480, 1485, 1490, 1495, 1500, 1505, 1510, 1515, 1520, 1525, 1530, 1535, 1540, 1545, 1550, 1555, 1560, 1565, 1570, 1575, 1580, 1585, 1590, 1595, 1600, 1605, 1610, 1615, 1620, 1625, 1630, 1635, 1640, 1645, 1650, 1655, 1660, 1665, 1670, 1675, 1680, 1685, 1690, 1695, 1700, 1705, 1710, 1715, 1720, 1725, 1730, 1735, 1740, 1745, 1750, 1755, 1760, 1765, 1770, 1775, 1780, 1785, 1790, 1795, 1800, 1805, 1810, 1815, 1820, 1825, 1830, 1835, 1840, 1845, 1850, 1855, 1860, 1865, 1870, 1875, 1880, 1885, 1890, 1895, 1900, 1905, 1910, 1915, 1920, 1925, 1930, 1935, 1940, 1945, 1950, 1955, 1960, 1965, 1970, 1975, 1980, 1985, 1990, 1995, 2000, 2010, 2015, 2020, 2025, 2030, 2035, 2040, 2045, 2050, 2055, 2060, 2065, 2070, 2075, 2080, 2085, 2090, 2095, 2100, 2105, 2110, 2115, 2120, 2125, 2130, 2135, 2140, 2145, 2150, 2155, 2160, 2165, 2170, 2175, 2180, 2185, 2190, 2195, 2200, 2205, 2210, 2215, 2220, 2225, 2230, 2235, 2240, 2245, 2250, 2255, 2260, 2265, 2270, 2275, 2280, 2285, 2290, 2295, 2300, 2305, 2310, 2315, 2320, 2325, 2330, 2335, 2340, 2345, 2350, 2355, 2360, 2365, 2370, 2375, 2380, 2385, 2390, 2395, 2400, 2405, 2410, 2415, 2420, 2425, 2430, 2435, 2440, 2445, 2450, 2455, 2460, 2465, 2470, 2475, 2480, 2485, 2490, 2495, 2500, 2505, 2510, 2515, 2520, 2525, 2530, 2535, 2540, 2545, 2550, 2555, 2560, 2565, 2570, 2575, 2580, 2585, 2590, 2595, 2600, 2605, 2610, 2615, 2620, 2625, 2630, 2635, 2640, 2645, 2650, 2655, 2660, 2665, 2670, 2675, 2680, 2685, 2690, 2695, 2700, 2705, 2710, 2715, 2720, 2725, 2730, 2735, 2740, 2745, 2750, 2755, 2760, 2765, 2770, 2775, 2780, 2785, 2790, 2795, 2800, 2805, 2810, 2815, 2820, 2825, 2830, 2835, 2840, 2845, 2850, 2855, 2860, 2865, 2870, 2875, 2880, 2885, 2890, 2895, 2900, 2905, 2910, 2915, 2920, 2925, 2930, 2935, 2940, 2945, 2950, 2955, 2960, 2965, 2970, 2975, 2980, 2985, 2990, 2995, 3000, 3010, 3015, 3020, 3025, 3030, 3035, 3040, 3045, 3050, 3055, 3060, 3065, 3070, 3075, 3080, 3085, 3090, 3095, 3100, 3105, 3110, 3115, 3120, 3125, 3130, 3135, 3140, 3145, 3150, 3155, 3160, 3165, 3170, 3175, 3180, 3185, 3190, 3195, 3200, 3205, 3210, 3215, 3220, 3225, 3230, 3235, 3240, 3245, 3250, 3255, 3260, 3265, 3270, 3275, 3280, 3285, 3290, 3295, 3300, 3305, 3310, 3315, 3320, 3325, 3330, 3335, 3340, 3345, 3350, 3355, 3360, 3365, 3370, 3375, 3380, 3385, 3390, 3395, 3400, 3405, 3410, 3415, 3420, 3425, 3430, 3435, 3440, 3445, 3450, 3455, 3460, 3465, 3470, 3475, 3480, 3485, 3490, 3495, 3500, 3505, 3510, 3515, 3520, 3525, 3530, 3535, 3540, 3545, 3550, 3555, 3560, 3565, 3570, 3575, 3580, 3585, 3590, 3595, 3600, 3605, 3610, 3615, 3620, 3625, 3630, 3635, 3640, 3645, 3650, 3655, 3660, 3665, 3670, 3675, 3680, 3685, 3690, 3695, 3700, 3705, 3710, 3715, 3720, 3725, 3730, 3735, 3740, 3745, 3750, 3755, 3760, 3765, 3770, 3775, 3780, 3785, 3790, 3795, 3800, 3805, 3810, 3815, 3820, 3825, 3830, 3835, 3840, 3845, 3850, 3855, 3860, 3865, 3870, 3875, 3880, 3885, 3890, 3895, 3900, 3905, 3910, 3915, 3920, 3925, 3930, 3935, 3940, 3945, 3950, 3955, 3960, 3965, 3970, 3975, 3980, 3985, 3990, 3995, 4000, 4010, 4015, 4020, 4025, 4030, 4035, 4040, 4045, 4050, 4055, 4060, 4065, 4070, 4075, 4080, 4085, 4090, 4095, 4100, 4105, 4110, 4115, 4120, 4125, 4130, 4135, 4140, 4145, 4150, 4155, 4160, 4165, 4170, 4175, 4180, 4185, 4190, 4195, 4200, 4205, 4210, 4215, 4220, 4225, 4230, 4235, 4240, 4245, 4250, 4255, 4260, 4265, 4270, 4275, 4280, 4285, 4290, 4295, 4300, 4305, 4310, 4315, 4320, 4325, 4330, 4335, 4340, 4345, 4350, 4355, 4360, 4365, 4370, 4375, 4380, 4385, 4390, 4395, 4400, 4405, 4410, 4415, 4420, 4425, 4430, 4435, 4440, 4445, 4450, 4455, 4460, 4465, 4470, 4475, 4480, 4485, 4490, 4495, 4500, 4505, 4510, 4515, 4520, 4525, 4530, 4535, 4540, 4545, 4550, 4555, 4560, 4565, 4570, 4575, 4580, 4585, 4590, 4595, 4600, 4605, 4610, 4615, 4620, 4625, 4630, 4635, 4640, 4645, 4650, 4655, 4660, 4665, 4670, 4675, 4680, 4685, 4690, 4695, 4700, 4705, 4710, 4715, 4720, 4725, 4730, 4735, 4740, 4745, 4750, 4755, 4760, 4765, 4770, 4775, 4780, 4785, 4790, 4795, 4800, 4805, 4810, 4815, 4820, 4825, 4830, 4835, 4840, 4845, 4850, 4855, 4860, 4865, 4870, 4875, 4880, 4885, 4890, 4895, 4900, 4905, 4910, 4915, 4920, 4925, 4930, 4935, 4940, 4945, 4950, 4955, 4960, 4965, 4970, 4975, 4980, 4985, 4990, 4995, 5000, 5005, 5010, 5015, 5020, 5025, 5030, 5035, 5040, 5045, 5050, 5055, 5060, 5065, 5070, 5075, 5080, 5085, 5090, 5095, 5100, 5105, 5110, 5115, 5120, 5125, 5130, 5135, 5140, 5145, 5150, 5155, 5160, 5165, 5170, 5175, 5180, 5185, 5190, 5195, 5200, 5205, 5210, 5215, 5220, 5225, 5230, 5235, 5240, 5245, 5250, 5255, 5260, 5265, 5270, 5275, 5280, 5285, 5290, 5295, 5300, 5305, 5310, 5315, 5320, 5325, 5330, 5335, 5340, 5345, 5350, 5355, 5360, 5365, 5370, 5375, 5380, 5385, 5390, 5395, 5400, 5405, 5410, 5415, 5420, 5425, 5430, 5435, 5440, 5445, 5450, 5455, 5460, 5465, 5470, 5475, 5480, 5485, 5490, 5495, 5500, 5505, 5510, 5515, 5520, 5525, 5530, 5535, 5540, 5545, 5550, 5555, 5560, 5565, 5570, 5575, 5580, 5585, 5590, 5595, 5600, 5605, 5610, 5615, 5620, 5625, 5630, 5635, 5640, 5645, 5650, 5655, 5660, 5665, 5670, 5675, 5680, 5685, 5690, 5695, 5700, 5705, 5710, 5715, 5720, 5725, 5730, 5735, 5740, 5745, 5750, 5755, 5760, 5765, 5770, 5775, 5780, 5785, 5790, 5795, 5800, 5805, 5810, 5815, 5820, 5825, 5830, 5835, 5840, 5845, 5850, 5855, 5860, 5865, 5870, 5875, 5880, 5885, 5890, 5895, 5900, 5905, 5910, 5915, 5920, 5925, 5930, 5935, 5940, 5945, 5950, 5955, 5960, 5965, 5970, 5975, 5980, 5985, 5990, 5995 and 6000 mg.

Preferably, for a body weight of 10-200 kg, the maintenance dosage level (6-25 mg/kg) of anti-thrombin aptamer is 60-5000 mg. Preferably, for a body weight of 10-50 kg, the maintenance dosage level is 60-1250 mg; for a body weight of 50-100 kg, the maintenance dosage level is 300-2500 mg; for a body weight of 100-150 kg, the maintenance dosage level is 600-3750 mg; and for a body weight of 150-200 kg, the maintenance dosage level is 900-5000 mg. The maintenance dose may be any one of 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995, 1000, 1005, 1010, 1015, 1020, 1025, 1030, 1035, 1040, 1045, 1050, 1055, 1060, 1065, 1070, 1075, 1080, 1085, 1090, 1095, 1100, 1105, 1110, 1115, 1120, 1125, 1130, 1135, 1140, 1145, 1150, 1155, 1160, 1165, 1170, 1175, 1180, 1185, 1190, 1195, 1200, 1205, 1210, 1215, 1220, 1225, 1230, 1235, 1240, 1245, 1250, 1255, 1260, 1265, 1270, 1275, 1280, 1285, 1290, 1295, 1300, 1305, 1310, 1315, 1320, 1325, 1330, 1335, 1340, 1345, 1350, 1355, 1360, 1365, 1370, 1375, 1380, 1385, 1390, 1395, 1400, 1405, 1410, 1415, 1420, 1425, 1430, 1435, 1440, 1445, 1450, 1455, 1460, 1465, 1470, 1475, 1480, 1485, 1490, 1495, 1500, 1505, 1510, 1515, 1520, 1525, 1530, 1535, 1540, 1545, 1550, 1555, 1560, 1565, 1570, 1575, 1580, 1585, 1590, 1595, 1600, 1605, 1610, 1615, 1620, 1625, 1630, 1635, 1640, 1645, 1650, 1655, 1660, 1665, 1670, 1675, 1680, 1685, 1690, 1695, 1700, 1705, 1710, 1715, 1720, 1725, 1730, 1735, 1740, 1745, 1750, 1755, 1760, 1765, 1770, 1775, 1780, 1785, 1790, 1795, 1800, 1805, 1810, 1815, 1820, 1825, 1830, 1835, 1840, 1845, 1850, 1855, 1860, 1865, 1870, 1875, 1880, 1885, 1890, 1895, 1900, 1905, 1910, 1915, 1920, 1925, 1930, 1935, 1940, 1945, 1950, 1955, 1960, 1965, 1970, 1975, 1980, 1985, 1990, 1995, 2000, 2010, 2015, 2020, 2025, 2030, 2035, 2040, 2045, 2050, 2055, 2060, 2065, 2070, 2075, 2080, 2085, 2090, 2095, 2100, 2105, 2110, 2115, 2120, 2125, 2130, 2135, 2140, 2145, 2150, 2155, 2160, 2165, 2170, 2175, 2180, 2185, 2190, 2195, 2200, 2205, 2210, 2215, 2220, 2225, 2230, 2235, 2240, 2245, 2250, 2255, 2260, 2265, 2270, 2275, 2280, 2285, 2290, 2295, 2300, 2305, 2310, 2315, 2320, 2325, 2330, 2335, 2340, 2345, 2350, 2355, 2360, 2365, 2370, 2375, 2380, 2385, 2390, 2395, 2400, 2405, 2410, 2415, 2420, 2425, 2430, 2435, 2440, 2445, 2450, 2455, 2460, 2465, 2470, 2475, 2480, 2485, 2490, 2495, 2500, 2505, 2510, 2515, 2520, 2525, 2530, 2535, 2540, 2545, 2550, 2555, 2560, 2565, 2570, 2575, 2580, 2585, 2590, 2595, 2600, 2605, 2610, 2615, 2620, 2625, 2630, 2635, 2640, 2645, 2650, 2655, 2660, 2665, 2670, 2675, 2680, 2685, 2690, 2695, 2700, 2705, 2710, 2715, 2720, 2725, 2730, 2735, 2740, 2745, 2750, 2755, 2760, 2765, 2770, 2775, 2780, 2785, 2790, 2795, 2800, 2805, 2810, 2815, 2820, 2825, 2830, 2835, 2840, 2845, 2850, 2855, 2860, 2865, 2870, 2875, 2880, 2885, 2890, 2895, 2900, 2905, 2910, 2915, 2920, 2925, 2930, 2935, 2940, 2945, 2950, 2955, 2960, 2965, 2970, 2975, 2980, 2985, 2990, 2995, 3000, 3010, 3015, 3020, 3025, 3030, 3035, 3040, 3045, 3050, 3055, 3060, 3065, 3070, 3075, 3080, 3085, 3090, 3095, 3100, 3105, 3110, 3115, 3120, 3125, 3130, 3135, 3140, 3145, 3150, 3155, 3160, 3165, 3170, 3175, 3180, 3185, 3190, 3195, 3200, 3205, 3210, 3215, 3220, 3225, 3230, 3235, 3240, 3245, 3250, 3255, 3260, 3265, 3270, 3275, 3280, 3285, 3290, 3295, 3300, 3305, 3310, 3315, 3320, 3325, 3330, 3335, 3340, 3345, 3350, 3355, 3360, 3365, 3370, 3375, 3380, 3385, 3390, 3395, 3400, 3405, 3410, 3415, 3420, 3425, 3430, 3435, 3440, 3445, 3450, 3455, 3460, 3465, 3470, 3475, 3480, 3485, 3490, 3495, 3500, 3505, 3510, 3515, 3520, 3525, 3530, 3535, 3540, 3545, 3550, 3555, 3560, 3565, 3570, 3575, 3580, 3585, 3590, 3595, 3600, 3605, 3610, 3615, 3620, 3625, 3630, 3635, 3640, 3645, 3650, 3655, 3660, 3665, 3670, 3675, 3680, 3685, 3690, 3695, 3700, 3705, 3710, 3715, 3720, 3725, 3730, 3735, 3740, 3745, 3750, 3755, 3760, 3765, 3770, 3775, 3780, 3785, 3790, 3795, 3800, 3805, 3810, 3815, 3820, 3825, 3830, 3835, 3840, 3845, 3850, 3855, 3860, 3865, 3870, 3875, 3880, 3885, 3890, 3895, 3900, 3905, 3910, 3915, 3920, 3925, 3930, 3935, 3940, 3945, 3950, 3955, 3960, 3965, 3970, 3975, 3980, 3985, 3990, 3995, 4000, 4010, 4015, 4020, 4025, 4030, 4035, 4040, 4045, 4050, 4055, 4060, 4065, 4070, 4075, 4080, 4085, 4090, 4095, 4100, 4105, 4110, 4115, 4120, 4125, 4130, 4135, 4140, 4145, 4150, 4155, 4160, 4165, 4170, 4175, 4180, 4185, 4190, 4195, 4200, 4205, 4210, 4215, 4220, 4225, 4230, 4235, 4240, 4245, 4250, 4255, 4260, 4265, 4270, 4275, 4280, 4285, 4290, 4295, 4300, 4305, 4310, 4315, 4320, 4325, 4330, 4335, 4340, 4345, 4350, 4355, 4360, 4365, 4370, 4375, 4380, 4385, 4390, 4395, 4400, 4405, 4410, 4415, 4420, 4425, 4430, 4435, 4440, 4445, 4450, 4455, 4460, 4465, 4470, 4475, 4480, 4485, 4490, 4495, 4500, 4505, 4510, 4515, 4520, 4525, 4530, 4535, 4540, 4545, 4550, 4555, 4560, 4565, 4570, 4575, 4580, 4585, 4590, 4595, 4600, 4605, 4610, 4615, 4620, 4625, 4630, 4635, 4640, 4645, 4650, 4655, 4660, 4665, 4670, 4675, 4680, 4685, 4690, 4695, 4700, 4705, 4710, 4715, 4720, 4725, 4730, 4735, 4740, 4745, 4750, 4755, 4760, 4765, 4770, 4775, 4780, 4785, 4790, 4795, 4800, 4805, 4810, 4815, 4820, 4825, 4830, 4835, 4840, 4845, 4850, 4855, 4860, 4865, 4870, 4875, 4880, 4885, 4890, 4895, 4900, 4905, 4910, 4915, 4920, 4925, 4930, 4935, 4940, 4945, 4950, 4955, 4960, 4965, 4970, 4975, 4980, 4985, 4990, 4995 and 5000 mg.

More preferably, for a body weight of 10-200 kg, the maintenance dosage level (8-18 mg/kg) of anti-thrombin aptamer is 80-3600 mg. Preferably, for a body weight of 10-50 kg, the maintenance dosage level is 80-900 mg; for a body weight of 50-100 kg, the maintenance dosage level is 400-1800 mg; for a body weight of 100-150, the maintenance dosage level is 800-2700 mg; and for a body weight of 150-200 kg, the total dosage level is 1200-3600 mg. The maintenance dose may be any one of 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995, 1000, 1005, 1010, 1015, 1020, 1025, 1030, 1035, 1040, 1045, 1050, 1055, 1060, 1065, 1070, 1075, 1080, 1085, 1090, 1095, 1100, 1105, 1110, 1115, 1120, 1125, 1130, 1135, 1140, 1145, 1150, 1155, 1160, 1165, 1170, 1175, 1180, 1185, 1190, 1195, 1200, 1205, 1210, 1215, 1220, 1225, 1230, 1235, 1240, 1245, 1250, 1255, 1260, 1265, 1270, 1275, 1280, 1285, 1290, 1295, 1300, 1305, 1310, 1315, 1320, 1325, 1330, 1335, 1340, 1345, 1350, 1355, 1360, 1365, 1370, 1375, 1380, 1385, 1390, 1395, 1400, 1405, 1410, 1415, 1420, 1425, 1430, 1435, 1440, 1445, 1450, 1455, 1460, 1465, 1470, 1475, 1480, 1485, 1490, 1495, 1500, 1505, 1510, 1515, 1520, 1525, 1530, 1535, 1540, 1545, 1550, 1555, 1560, 1565, 1570, 1575, 1580, 1585, 1590, 1595, 1600, 1605, 1610, 1615, 1620, 1625, 1630, 1635, 1640, 1645, 1650, 1655, 1660, 1665, 1670, 1675, 1680, 1685, 1690, 1695, 1700, 1705, 1710, 1715, 1720, 1725, 1730, 1735, 1740, 1745, 1750, 1755, 1760, 1765, 1770, 1775, 1780, 1785, 1790, 1795, 1800, 1805, 1810, 1815, 1820, 1825, 1830, 1835, 1840, 1845, 1850, 1855, 1860, 1865, 1870, 1875, 1880, 1885, 1890, 1895, 1900, 1905, 1910, 1915, 1920, 1925, 1930, 1935, 1940, 1945, 1950, 1955, 1960, 1965, 1970, 1975, 1980, 1985, 1990, 1995, 2000, 2010, 2015, 2020, 2025, 2030, 2035, 2040, 2045, 2050, 2055, 2060, 2065, 2070, 2075, 2080, 2085, 2090, 2095, 2100, 2105, 2110, 2115, 2120, 2125, 2130, 2135, 2140, 2145, 2150, 2155, 2160, 2165, 2170, 2175, 2180, 2185, 2190, 2195, 2200, 2205, 2210, 2215, 2220, 2225, 2230, 2235, 2240, 2245, 2250, 2255, 2260, 2265, 2270, 2275, 2280, 2285, 2290, 2295, 2300, 2305, 2310, 2315, 2320, 2325, 2330, 2335, 2340, 2345, 2350, 2355, 2360, 2365, 2370, 2375, 2380, 2385, 2390, 2395, 2400, 2405, 2410, 2415, 2420, 2425, 2430, 2435, 2440, 2445, 2450, 2455, 2460, 2465, 2470, 2475, 2480, 2485, 2490, 2495, 2500, 2505, 2510, 2515, 2520, 2525, 2530, 2535, 2540, 2545, 2550, 2555, 2560, 2565, 2570, 2575, 2580, 2585, 2590, 2595, 2600, 2605, 2610, 2615, 2620, 2625, 2630, 2635, 2640, 2645, 2650, 2655, 2660, 2665, 2670, 2675, 2680, 2685, 2690, 2695, 2700, 2705, 2710, 2715, 2720, 2725, 2730, 2735, 2740, 2745, 2750, 2755, 2760, 2765, 2770, 2775, 2780, 2785, 2790, 2795, 2800, 2805, 2810, 2815, 2820, 2825, 2830, 2835, 2840, 2845, 2850, 2855, 2860, 2865, 2870, 2875, 2880, 2885, 2890, 2895, 2900, 2905, 2910, 2915, 2920, 2925, 2930, 2935, 2940, 2945, 2950, 2955, 2960, 2965, 2970, 2975, 2980, 2985, 2990, 2995, 3000, 3010, 3015, 3020, 3025, 3030, 3035, 3040, 3045, 3050, 3055, 3060, 3065, 3070, 3075, 3080, 3085, 3090, 3095, 3100, 3105, 3110, 3115, 3120, 3125, 3130, 3135, 3140, 3145, 3150, 3155, 3160, 3165, 3170, 3175, 3180, 3185, 3190, 3195, 3200, 3205, 3210, 3215, 3220, 3225, 3230, 3235, 3240, 3245, 3250, 3255, 3260, 3265, 3270, 3275, 3280, 3285, 3290, 3295, 3300, 3305, 3310, 3315, 3320, 3325, 3330, 3335, 3340, 3345, 3350, 3355, 3360, 3365, 3370, 3375, 3380, 3385, 3390, 3395, 3400, 3405, 3410, 3415, 3420, 3425, 3430, 3435, 3440, 3445, 3450, 3455, 3460, 3465, 3470, 3475, 3480, 3485, 3490, 3495, 3500, 3505, 3510, 3515, 3520, 3525, 3530, 3535, 3540, 3545, 3550, 3555, 3560, 3565, 3570, 3575, 3580, 3585, 3590, 3595 and 3600 mg.

Even more preferably, for a body weight of 10-200 kg, the maintenance dosage level (10-16 mg/kg) of anti-thrombin aptamer is 100-3200 mg. Preferably, for a body weight of 10-50 kg, the maintenance dosage level is 100-800 mg; for a body weight of 50-100 kg, the maintenance dosage level is 500-1600 mg; for a body weight of 100-150 kg, the maintenance dosage level is 1000-2400 mg; and for a body weight of 150-200 kg, the maintenance dosage level is 1500-3200 mg. The maintenance dose may be any one of 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995, 1000, 1005, 1010, 1015, 1020, 1025, 1030, 1035, 1040, 1045, 1050, 1055, 1060, 1065, 1070, 1075, 1080, 1085, 1090, 1095, 1100, 1105, 1110, 1115, 1120, 1125, 1130, 1135, 1140, 1145, 1150, 1155, 1160, 1165, 1170, 1175, 1180, 1185, 1190, 1195, 1200, 1205, 1210, 1215, 1220, 1225, 1230, 1235, 1240, 1245, 1250, 1255, 1260, 1265, 1270, 1275, 1280, 1285, 1290, 1295, 1300, 1305, 1310, 1315, 1320, 1325, 1330, 1335, 1340, 1345, 1350, 1355, 1360, 1365, 1370, 1375, 1380, 1385, 1390, 1395, 1400, 1405, 1410, 1415, 1420, 1425, 1430, 1435, 1440, 1445, 1450, 1455, 1460, 1465, 1470, 1475, 1480, 1485, 1490, 1495, 1500, 1505, 1510, 1515, 1520, 1525, 1530, 1535, 1540, 1545, 1550, 1555, 1560, 1565, 1570, 1575, 1580, 1585, 1590, 1595, 1600, 1605, 1610, 1615, 1620, 1625, 1630, 1635, 1640, 1645, 1650, 1655, 1660, 1665, 1670, 1675, 1680, 1685, 1690, 1695, 1700, 1705, 1710, 1715, 1720, 1725, 1730, 1735, 1740, 1745, 1750, 1755, 1760, 1765, 1770, 1775, 1780, 1785, 1790, 1795, 1800, 1805, 1810, 1815, 1820, 1825, 1830, 1835, 1840, 1845, 1850, 1855, 1860, 1865, 1870, 1875, 1880, 1885, 1890, 1895, 1900, 1905, 1910, 1915, 1920, 1925, 1930, 1935, 1940, 1945, 1950, 1955, 1960, 1965, 1970, 1975, 1980, 1985, 1990, 1995, 2000, 2010, 2015, 2020, 2025, 2030, 2035, 2040, 2045, 2050, 2055, 2060, 2065, 2070, 2075, 2080, 2085, 2090, 2095, 2100, 2105, 2110, 2115, 2120, 2125, 2130, 2135, 2140, 2145, 2150, 2155, 2160, 2165, 2170, 2175, 2180, 2185, 2190, 2195, 2200, 2205, 2210, 2215, 2220, 2225, 2230, 2235, 2240, 2245, 2250, 2255, 2260, 2265, 2270, 2275, 2280, 2285, 2290, 2295, 2300, 2305, 2310, 2315, 2320, 2325, 2330, 2335, 2340, 2345, 2350, 2355, 2360, 2365, 2370, 2375, 2380, 2385, 2390, 2395, 2400, 2405, 2410, 2415, 2420, 2425, 2430, 2435, 2440, 2445, 2450, 2455, 2460, 2465, 2470, 2475, 2480, 2485, 2490, 2495, 2500, 2505, 2510, 2515, 2520, 2525, 2530, 2535, 2540, 2545, 2550, 2555, 2560, 2565, 2570, 2575, 2580, 2585, 2590, 2595, 2600, 2605, 2610, 2615, 2620, 2625, 2630, 2635, 2640, 2645, 2650, 2655, 2660, 2665, 2670, 2675, 2680, 2685, 2690, 2695, 2700, 2705, 2710, 2715, 2720, 2725, 2730, 2735, 2740, 2745, 2750, 2755, 2760, 2765, 2770, 2775, 2780, 2785, 2790, 2795, 2800, 2805, 2810, 2815, 2820, 2825, 2830, 2835, 2840, 2845, 2850, 2855, 2860, 2865, 2870, 2875, 2880, 2885, 2890, 2895, 2900, 2905, 2910, 2915, 2920, 2925, 2930, 2935, 2940, 2945, 2950, 2955, 2960, 2965, 2970, 2975, 2980, 2985, 2990, 2995, 3000, 3010, 3015, 3020, 3025, 3030, 3035, 3040, 3045, 3050, 3055, 3060, 3065, 3070, 3075, 3080, 3085, 3090, 3095, 3100, 3105, 3110, 3115, 3120, 3125, 3130, 3135, 3140, 3145, 3150, 3155, 3160, 3165, 3170, 3175, 3180, 3185, 3190, 3195 and 3200 mg.

Most preferably, for a body weight of 10-200 kg, the maintenance dosage level (12-14 mg/kg) of anti-thrombin aptamer is 120-2800 mg. Preferably, for a body weight of 10-50 kg, the maintenance dosage level is 120-700 mg; for a body weight of 50-100 kg, the maintenance dosage level is 600-1400 mg; for a body weight of 100-150 kg, the maintenance dosage level is 1200-2100 mg; and for a body weight of 150-200 kg, the maintenance dosage level is 1800-2800 mg. The maintenance dose may be any one of 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995, 1000, 1005, 1010, 1015, 1020, 1025, 1030, 1035, 1040, 1045, 1050, 1055, 1060, 1065, 1070, 1075, 1080, 1085, 1090, 1095, 1100, 1105, 1110, 1115, 1120, 1125, 1130, 1135, 1140, 1145, 1150, 1155, 1160, 1165, 1170, 1175, 1180, 1185, 1190, 1195, 1200, 1205, 1210, 1215, 1220, 1225, 1230, 1235, 1240, 1245, 1250, 1255, 1260, 1265, 1270, 1275, 1280, 1285, 1290, 1295, 1300, 1305, 1310, 1315, 1320, 1325, 1330, 1335, 1340, 1345, 1350, 1355, 1360, 1365, 1370, 1375, 1380, 1385, 1390, 1395, 1400, 1405, 1410, 1415, 1420, 1425, 1430, 1435, 1440, 1445, 1450, 1455, 1460, 1465, 1470, 1475, 1480, 1485, 1490, 1495, 1500, 1505, 1510, 1515, 1520, 1525, 1530, 1535, 1540, 1545, 1550, 1555, 1560, 1565, 1570, 1575, 1580, 1585, 1590, 1595, 1600, 1605, 1610, 1615, 1620, 1625, 1630, 1635, 1640, 1645, 1650, 1655, 1660, 1665, 1670, 1675, 1680, 1685, 1690, 1695, 1700, 1705, 1710, 1715, 1720, 1725, 1730, 1735, 1740, 1745, 1750, 1755, 1760, 1765, 1770, 1775, 1780, 1785, 1790, 1795, 1800, 1805, 1810, 1815, 1820, 1825, 1830, 1835, 1840, 1845, 1850, 1855, 1860, 1865, 1870, 1875, 1880, 1885, 1890, 1895, 1900, 1905, 1910, 1915, 1920, 1925, 1930, 1935, 1940, 1945, 1950, 1955, 1960, 1965, 1970, 1975, 1980, 1985, 1990, 1995, 2000, 2010, 2015, 2020, 2025, 2030, 2035, 2040, 2045, 2050, 2055, 2060, 2065, 2070, 2075, 2080, 2085, 2090, 2095, 2100, 2105, 2110, 2115, 2120, 2125, 2130, 2135, 2140, 2145, 2150, 2155, 2160, 2165, 2170, 2175, 2180, 2185, 2190, 2195, 2200, 2205, 2210, 2215, 2220, 2225, 2230, 2235, 2240, 2245, 2250, 2255, 2260, 2265, 2270, 2275, 2280, 2285, 2290, 2295, 2300, 2305, 2310, 2315, 2320, 2325, 2330, 2335, 2340, 2345, 2350, 2355, 2360, 2365, 2370, 2375, 2380, 2385, 2390, 2395, 2400, 2405, 2410, 2415, 2420, 2425, 2430, 2435, 2440, 2445, 2450, 2455, 2460, 2465, 2470, 2475, 2480, 2485, 2490, 2495, 2500, 2505, 2510, 2515, 2520, 2525, 2530, 2535, 2540, 2545, 2550, 2555, 2560, 2565, 2570, 2575, 2580, 2585, 2590, 2595, 2600, 2605, 2610, 2615, 2620, 2625, 2630, 2635, 2640, 2645, 2650, 2655, 2660, 2665, 2670, 2675, 2680, 2685, 2690, 2695, 2700, 2705, 2710, 2715, 2720, 2725, 2730, 2735, 2740, 2745, 2750, 2755, 2760, 2765, 2770, 2775, 2780, 2785, 2790, 2795 and 2800 mg.

In specific embodiments, the formulations are dosed and administered according to the following Tables 1, 2 and 3.

TABLE 1

| | Target | | | | |
|---|---|---|---|---|---|
| | [plasma] | 10 μg/ml | 15 μg/ml | 20 μg/ml | 25 μg/ml |
| | Total dose | 8.33 mg/kg | 11.83 mg/kg | 15.73 mg/kg | 19.83 mg/kg |
| Infusion regimen | loading dose | 1.33 mg/kg | 1.33 mg/kg | 1.33 mg/kg | 1.33 mg/kg |
| (mg/kg) | | | | | |
| Infusion regimen | loading dose | 0.5 | 0.5 | 0.5 | 0.5 |
| (mg/kg/min) | | | | | |
| Infusion regimen | maintenance | 7.0 mg/kg | 10.5 mg/kg | 14.4 mg/kg | 18.5 mg/kg |
| (mg/kg) | dose | | | | |
| Infusion regimen | maintenance | 0.0292 | 0.0438 | 0.0600 | 0.0771 |
| (mg/kg/min) | dose | | | | |

TABLE 2

| | Target | | | | |
|---|---|---|---|---|---|
| | [plasma] | 10 μg/ml | 15 μg/ml | 20 μg/ml | 25 μg/ml |
| | Total dose | 8.75 mg/kg | 12.40 mg/kg | 16.00 mg/kg | 19.80 mg/kg |
| Infusion regimen | loading dose | 2.0 mg/kg | 2.0 mg/kg | 2.0 mg/kg | 2.0 mg/kg |
| (mg/kg) | | | | | |
| Infusion regimen | loading dose | 0.5 | 0.5 | 0.5 | 0.5 |
| (mg/kg/min) | | | | | |
| Infusion regimen | maintenance | 6.75 mg/kg | 10.4 mg/kg | 14.0 mg/kg | 17.8 mg/kg |
| (mg/kg) | dose | | | | |
| Infusion regimen | maintenance | 0.0281 | 0.0433 | 0.0583 | 0.0742 |
| (mg/kg/min) | dose | | | | |

TABLE 3

| | Target | | | | |
|---|---|---|---|---|---|
| | [plasma] | 10 μg/ml | 15 μg/ml | 20 μg/ml | 25 μg/ml |
| | Total dose | 7.95 mg/kg | 11.85 mg/kg | 15.85 mg/kg | 19.80 mg/kg |
| Infusion regimen | loading dose | 0.75 mg/kg | 0.85 mg/kg | 1.05 mg/kg | 2.0 mg/kg |
| (mg/kg) | | | | | |
| Infusion regimen | loading dose | 0.5 | 0.5 | 0.5 | 0.5 |
| (mg/kg/min) | | | | | |
| Infusion regimen | maintenance | 7.2 mg/kg | 11.0 mg/kg | 14.8 mg/kg | 17.8 mg/kg |
| (mg/kg) | dose | | | | |
| Infusion regimen | maintenance | 0.0300 | 0.0458 | 0.0617 | 0.0742 |
| (mg/kg/min) | dose | | | | |

The formulations may be administered to a vertebrate, preferably a mammal, and more preferably a human. The terms “patient” and “subject” are used interchangeably throughout the application, and these terms include both human and veterinary subjects. The aptamer formulations provided herein are administered to subjects, particularly, human subjects, in an amount effective to inhibit, reduce, block or otherwise modulate thrombin-mediated coagulation.

### Indications

The formulations are used to treat, prevent or ameliorate thrombin-mediated diseases and disorders. The diseases and disorders to be treated, prevented or ameliorated are selected from the group consisting of: renal impairment, impaired hepatic function and heparin induced thrombocytopenia. Aptamers of the invention also provide a safe and effective modality for modulating coagulation, particularly for anticoagulation in relation to kidney dialysis and surgical procedures, such as percutaneous coronary intervention (PCI), stent placement surgery, peripheral arterial occlusion disease (PAOD), cardiopulmonary bypass procedures, coronary artery bypass graft (CABG) surgery, angioplasty, cardiovascular and peripheral vascular open and endovascular surgery, heart valve replacement surgery, and surgery to treat coronary disease and/or vascular disease in veins or arteries.

### Therapeutic Rationale

Without wishing to be bound by theory regarding mechanism of action, the following therapeutic rationale is offered by way of example only.

ARC2172 is a single-stranded DNA aptamer that binds to exosite I on thrombin. ARC2172 is an anti-thrombin aptamer with potent anticoagulant properties and rapid onset and offset of action. In addition, ARC2172 does not require an antidote for reversal of activity.

### Combination Therapy

One embodiment of the invention comprises a formulation of the invention used in combination with one or more other treatments for coagulation related disorders. The aptamer formulation of the invention may contain, for example, more than one aptamer, e.g., an anti-thrombin aptamer and an aptamer against a clotting factor or a coagulation cascade protein. In some embodiments, an aptamer formulation of the invention, containing one or more aptamers, is administered in combination with another useful formulation or drug. In another embodiment, the aptamer formulations are used in combination with non-drug therapies or treatments, such as surgical procedures. In general, the currently available dosage forms of the known therapeutic agents and the uses of non-drug therapies for use in such combinations will be suitable.

“Combination therapy” (or “co-therapy”) includes the administration of an aptamer formulation of the invention and at least a second agent or treatment as part of a specific treatment regimen intended to provide the beneficial effect from the co-action of these therapeutic agents or treatments. The beneficial effect of the combination includes, but is not limited to, pharmacokinetic or pharmacodynamic co-action resulting from the combination of therapeutic agent or treatments. Administration of these therapeutic agents or treatments in combination typically is carried out over a defined time period (usually minutes, hours, days or weeks depending upon the combination selected).

“Combination therapy” may, but generally is not, intended to encompass the administration of two or more of these therapeutic agents or treatments as part of separate monotherapy regimens that incidentally and arbitrarily results in the combinations of the present invention. “Combination therapy” is intended to embrace administration of these therapeutic agents or treatments in a sequential manner, that is, wherein each therapeutic agent or treatment is administered at a different time, as well as administration of these therapeutic agents or treatments, or at least two of the therapeutic agents or treatments, in a substantially simultaneous manner. Substantially simultaneous administration can be accomplished, for example, by administering to the subject a single injection having a fixed ratio of each therapeutic agent or in multiple, single injections for each of the therapeutic agents.

Sequential or substantially simultaneous administration of each therapeutic agent or treatment can be effected by any appropriate route including, but not limited to, topical routes, oral routes, intravenous routes, subcutaneous, intramuscular routes, and direct absorption through mucous membrane tissues. The therapeutic agents or treatments can be administered by the same route or by different routes. For example, a first therapeutic agent or treatment of the combination selected may be administered by injection while the other therapeutic agents or treatments of the combination may be administered subcutaneously. Alternatively, for example, all therapeutic agents or treatments may be administered subcutaneously or all therapeutic agents or treatments may be administered by injection. The sequence in which the therapeutic agents or treatments are administered is not critical unless noted otherwise.

“Combination therapy” also can embrace the administration of the therapeutic agent or treatments as described above in further combination with other biologically active ingredients. Where the combination therapy comprises a non-drug treatment, the non-drug treatment may be conducted at any suitable time so long as a beneficial effect from the co-action of the combination of the therapeutic agent and non-drug treatment is achieved. For example, in appropriate cases, the beneficial effect is still achieved when the non-drug treatment is temporally removed from the administration of the therapeutic agent, perhaps by days or even weeks.

The formulations may be administered in combination with other drugs or therapies. For example, the formulations of the invention may be used in combination with heparin, aspirin, or other anticoagulants or anti-clotting agents.

### Packaging

The formulations can be packaged for use in a variety of pharmaceutically acceptable containers using any pharmaceutically acceptable container closure, as the formulations are compatible with PVC-containing and PVC-free containers and container closures. Examples of pharmaceutically acceptable containers include, but are not limited to, ampules, pre-filled syringes, intravenous bags, intravenous bottles and admix bags. For example, the formulation may be an aqueous formulation containing both anti-thrombin aptamer and pharmaceutically acceptable solvent. Alternatively, the formulation may contain lyophilized anti-thrombin aptamer in one compartment of an admix bag and a pharmaceutically acceptable solvent in a separate compartment of the admix bag such that the two compartments may be mixed together prior to administration to a patient. Pharmaceutically acceptable containers are well known in the art and commercially available. Preferably, the formulations are stored in a Type 1 glass vial with a butyl rubber stopper. The formulations in liquid form must be stored in a refrigerated environment. Preferably, the liquid formulations are stored at 2-8° C. Alternatively, the lyophililized formulations may be stored at room temperature, or refrigerated or frozen.

Preferably, the formulations are sterile. A “sterile” formulation, as used herein, means a formulation that has been brought to a state of sterility and has not been subsequently exposed to microbiological contamination, i.e., the container holding the sterile composition has not been compromised. Sterile compositions are generally prepared by pharmaceutical manufacturers in accordance with current Good Manufacturing Practice (“cGMP”) regulations of the U.S. Food and Drug Administration.

Procedures for filling pharmaceutical formulations in pharmaceutically acceptable containers, and their subsequent processing are known in the art. These procedures can be used to produce sterile pharmaceutical drug products often required for health care. See, e.g., Center for Drug Evaluation and Research (CDER) and Center for Veterinary Medicine (CVM), “Guidance for Industry for the Submission Documentation for Sterilization Process Validation in Applications for Human and Veterinary Drug Products”, (November 1994). Examples of suitable procedures for producing sterile pharmaceutical drug products include, but are not limited to, terminal moist heat sterilization, ethylene oxide, radiation (i.e., gamma and electron beam) and aseptic processing techniques. Any one of these sterilization procedures can be used to produce the sterile pharmaceutical formulations described herein.

In some embodiments, sterile pharmaceutical formulations can be prepared using aseptic processing techniques. Sterility is maintained by using sterile materials and a controlled working environment. All containers and apparatus are sterilized, preferably by heat sterilization, prior to filling. Then, the container is filled under aseptic conditions, such as by passing the composition through a filter and filling the units. Therefore, the formulations can be sterile filled into a container to avoid the heat stress of terminal sterilization.

In some embodiments, the formulations are terminally sterilized using moist heat. Terminal sterilization can be used to destroy all viable microorganisms within the final, sealed container containing the pharmaceutical formulation. An autoclave is typically used to accomplish terminal heat-sterilization of drug products in their final packaging. Typical autoclave cycles in the pharmaceutical industry to achieve terminal sterilization of the final product are 121° C. for at least 10 minutes.

### Kits

The formulations may also be packaged in a kit. The kit will contain the formulation, along with instructions regarding administration of the drug. The kit may also contain one or more of the following: a syringe, an intravenous bag or bottle, the same drug in a different dosage form, or another drug. For example, the kit may contain both an intravenous formulation and a subcutaneous formulation of the present invention. Alternatively, the kit may contain lyophilized anti-thrombin aptamer and an intravenous bag of solution. The kit form is particularly advantageous when the separate components must be administered in different dosage forms (i.e., parenteral and oral) or are administered at different dosage intervals.

Preferably, the kits are stored at 5±3° C. The kits can also be stored at room temperature or frozen at −20° C.

Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled.

### EXAMPLES

The following examples, including the experiments conducted and results achieved are provided for illustrative purposes only and are not to be construed as limiting upon the present invention.

For all of the following examples, ARC2172 was provided in a formulation comprising ARC2172 and isotonic saline.

### Example 1

### Phase 1a Proof of Concept Trial

The single-center, Phase 1a trial examined the safety, tolerability and pharmacokinetics of escalating bolus doses of NU172 in 30 normal, healthy volunteers. Volunteers were given a single bolus dose of either 0.20, 0.43, 0.66, 1.32 or 2.00 mg/kg of NU172. In the trial, NU172 produced dose-dependent increases in anticoagulation, as measured by ACT, ECT, PT and PTT. The 2.00 mg/kg bolus dose of NU172 achieved target ACTs of approximately 400 seconds. Upon withdrawal of NU172, the ACT showed a rapid return toward baseline with a plasma half-life of approximately 10 minutes.

### Example 2

### Phase 1b Proof of Concept Trial

The single-center, Phase 1b trial examined the safety, tolerability and pharmacokinetics of intravenous bolus plus infusion dosing of NU172, in 24 healthy male volunteers. Volunteers were given a 2 mg/kg bolus dose followed by escalating infusion doses of NU172 for four hours. In all four cohorts, NU172 produced dose-dependent increases in anticoagulation, measured by activated clotting time (ACT), prothrombin time (PT) and activated partial thromboplastin time (aPTT). The highest infusion dose rate tested, 6.0 mg/kg/hr, resulted in an average ACT per subject ranging from 373 to 414 seconds and an increase of approximately three times baseline. Average PT values per subject ranged from 56 to 92 seconds and had an increase of approximately five times baseline. Average aPTT values per subject ranged from 130 to 178 seconds and had an increase of approximately five times baseline. All measurements were maintained stably throughout the four-hour infusion. Once the infusion ended, the ACT and other coagulation parameters showed a rapid return toward baseline, consistent with the short plasma half-life of NU172 observed in the Phase 1a trial. In addition, NU172 was well-tolerated with no serious adverse events.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for treating, preventing or ameliorating a thrombin mediated coagulation related disease or disorder comprising administering to a subject in need thereof a therapeutically effective dose of an aptamer that binds to thrombin and has the following structure: CGCCTAGGTTGGGTAGGGTGGTGGCG (SEQ ID NO: 1),
wherein the aptamer is administered at a dose in the range of 1.0 mg/kg to 30.0 mg/kg.

2. The method of claim 1, wherein the disease or disorder is selected from the group consisting of: renal impairment, impaired hepatic function and heparin induced thrombocytopenia.

3. The method of claim 1, wherein the administration is intravenous.

4. The method of claim 1, wherein the dose is 15 mg/kg.

5. The method of claim 1, wherein the dose achieves a steady state blood concentration of 10-35 μg/ml.

6. The method of claim 1, wherein the formulation is administered in combination with another drug.

7. The method of claim 6, wherein drug is selected from the group consisting of: heparin and aspirin.

8. The method of claim 1, wherein the formulation is administered to a subject before, during or after a surgical procedure.

9. The method of claim 8, wherein the surgical procedure is selected from the group consisting of: percutaneous coronary intervention (PCI), stent placement surgery, peripheral arterial occlusion disease (PAOD) surgery, cardiopulmonary bypass procedures, coronary artery bypass graft (CABG) surgery, angioplasty, cardiovascular and peripheral vascular open and endovascular surgery, heart valve replacement surgery, and surgery to treat coronary disease and/or vascular disease in veins or arteries.

10. A formulation comprising:
a) an aptamer that binds to thrombin comprising the following structure: CGCCTAGGTTGGGTAGGGTGGTGGCG (SEQ ID NO: 1); and
b) a pharmaceutically acceptable solvent,
wherein the formulation comprises 10-6000 mg of aptamer.

11. The formulation of claim 10, wherein the formulation comprises 20 ml of solvent.

12. The formulation of claim 10, wherein the formulation comprises 15 mg of aptamer per 1 ml of solvent.

13. The formulation of claim 10, wherein the formulation is aqueous.

14. The formulation of claim 10, wherein the aptamer is lyophilized.

15. The formulation of claim 10, wherein the formulation is packaged in a pharmaceutically acceptable container.

16. A kit comprising the formulation of claim 10.

17. Use of an aptamer that binds to thrombin and has the following structure: CGCCTAGGTTGGGTAGGGTGGTGGCG (SEQ ID NO: 1), in the manufacture of a medicament for treating, preventing or ameliorating a thrombin mediated coagulation related disease or disorder in a subject, wherein the aptamer is administered at a dose in the range of 1.0 mg/kg to 30.0 mg/kg.


SEQUENCE LISTING (5 sequences)

SEQ ID NO: 1 - DNA, 26 bp, Artificial Sequence
  Synthetic aptamer  ARC2172
cgcctaggttgggtagggtggtggcg

SEQ ID NO: 2 - DNA, 28 bp, Artificial Sequence
  Synthetic aptamer  ARC2171
ctgcctaggttgggtagggtggtggcag

SEQ ID NO: 3 - DNA, 30 bp, Artificial Sequence
  Synthetic aptamer  ARC2170
gctgcctaggttgggtagggtggtggcagc

SEQ ID NO: 4 - DNA, 30 bp, Artificial Sequence
  Synthetic aptamer  ARC2169
actgcctaggttgggtagggtggtggcagt

SEQ ID NO: 5 - DNA, 22 bp, Artificial Sequence
  Synthetic aptamer  ARCminimer
cctaggttgggtagggtggtgg